(19) United States
(12) Patent Application Publication
(10) Pub. No.: US 2026/0337672 A1
(43) Pub. Date: Oct. 29, 2026

(54) NON-INVASIVE METHODS OF ASSESSING TRANSPLANT REJECTION IN PREGNANT TRANSPLANT RECIPIENTS

(71) Applicant: Natera, Inc., San Carlos, CA (US)

(72) Inventors: Jessica TANG, San Jose, CA (US); Kate WOODRUFF, Santa Rosa, CA (US); Phikhanh VU, San Jose, CA (US); Chris SOTTO, San Carlos, CA (US); Ebad AHMED, San Jose, CA (US); Paul VAN HUMMELEN, San Francisco, CA (US); Gabrielle HEILEK, San Carlos, CA (US); Bernhard ZIMMERMANN, Manteca, CA (US); Hossein TABRIZIANI, San Diego, CA (US)

(73) Assignee: Natera, Inc., San Carlos, CA (US)

(21) Appl. No.: 19/117,611

(22) PCT Filed: Sep. 20, 2023
(86) PCT No.: PCT/US23/33299

§ 371 Date: Apr. 1, 2025

Related U.S. Application Data
(60) Provisional application No. 63/413,750, filed on Oct. 6, 2022.
(60) Provisional application No. 63/440,251, filed on Jan. 20, 2023.
(60) Provisional application No. 63/530,828, filed on Aug. 4, 2023.

(51) Int. Cl. C12Q 1/6851; C12N 15/10; C12Q 1/6806; C12Q 1/6874; G16B 20/20

(52) U.S. Cl. CPC C12Q 1/6851; C12N 15/1093; C12Q 1/6806; C12Q 1/6874; G16B 20/20

(57) ABSTRACT

The present disclosure provides methods for preparation and analysis of biological samples of maternal transplant recipients, wherein the methods comprise extracting cell-free DNA from the recipient, wherein the cell-free DNA comprises donor-derived cell-free DNA, recipient-derived cell-free DNA, and fetal-derived cell-free DNA, and measuring amounts of cell-free DNA and donor-derived cell-free DNA enables assessment of transplant rejection. The detection of the donor-derived cell-free DNA may be performed based on loci at which the maternal transplant recipient and biological father of the fetus are homozygous and the transplant donor is heterozygous.

### BACKGROUND

Rapid detection of graft injury and/or rejection remains a challenge for transplant recipients. This challenge increases in pregnant mothers as renal dysfunction and chronic hypertension are common in pregnant kidney transplant recipients. Distinguishing between transplant rejection, kidney disease progression, and preeclampsia in a non-invasive manner can be difficult in this group.

Following transplantation, tests for determining and monitoring transplant injury and/or rejection is a critical aspect of the post-transplantation care and determination of need for personalized immunosuppressive therapy. As well as being dangerous to the mother and fetus, conventional biopsy-based tests are invasive and costly and possibly lead to late diagnosis of transplant injury and/or rejection. Therefore, there is a need for a non-invasive test for transplant injury and/or rejection that is safer, more sensitive, and more specific than conventional biopsy-based tests to enable early diagnosis of transplant injury and/or rejection in the pregnant transplant recipient.

Thus, improved methods are needed to accurately diagnose, screen, test, and monitor transplant injury and/or rejection at an early stage in pregnant transplant recipients. The present disclosure provides this need.

### SUMMARY

In one aspect, the present disclosure relates to a method of preparing a composition of amplified DNA derived from a biological sample of a maternal transplant recipient useful for determination of transplant status, comprising: extracting cell-free DNA (cfDNA) from the biological sample of the maternal transplant recipient, wherein the extracted cfDNA comprises donor-derived cell-free DNA (dd-cfDNA) from the transplant, recipient-derived cell-free DNA (rd-cfDNA) from the maternal transplant recipient, and fetal-derived cell-free DNA (fd-cfDNA) from a fetus; preparing a composition from the cfDNA wherein a plurality of target loci are enriched, wherein the target loci comprise one or more SNP loci at which the maternal transplant recipient and biological father of the fetus are homozygous to ensure homozygosity of the fetus at the SNP loci, such that heterozygosity observed in the extracted cfDNA at the SNP loci originates from the transplant; and quantifying the amount of dd-cfDNA based on heterozygosity at the SNP loci to determine whether the amount of dd-cfDNA or a function thereof exceeds a cutoff threshold indicating transplant rejection.

In some embodiments, quantifying the cfDNA and dd-cfDNA comprises preparing a sequencing library from the extracted cfDNA and sequencing the sequencing library by high-throughput sequencing to obtain sequencing reads.

In some embodiments, the method further comprises sequencing paternal DNA of the biological father of the fetus, and identifying one or more SNP loci at which the maternal transplant recipient and biological father of the fetus are homozygous while the transplant or dd-cfDNA comprises a heterozygous allele. The amount of sequence reads derived from the heterozygous alleles at the aforementioned SNP loci can therefore be used to quantify the amount of dd-cfDNA in the biological sample of the pregnant transplant recipient.

In some embodiments, the target loci comprises 10-50,000 target loci, or 100-20,000target loci, or 100-1,000 target loci, or 1,000-10,000 target loci, or 10,000-50,000 target loci. In some embodiments, the method further comprises enriching the target loci by performing multiplex targeted amplification of the DNA at the 10-50,000 target loci, or the 100-20,000target loci, or the 100-1,000 target loci, or the 1,000-10,000 target loci, or the 10,000-50,000target loci, preferably in a single reaction volume. In some embodiments, the method further comprises enriching the target loci using hybrid capture probes targeting the 10-50,000 target loci, or the 100-20,000 target loci, or the 100-1,000 target loci, or the 1,000-10,000 target loci, or the 10,000-50,000 target loci, preferably in a single reaction volume.

In one aspect, the present disclosure relates to a method of preparing a composition of DNA derived from a biological sample of a maternal transplant recipient useful for determination of transplant rejection, wherein the composition of DNA comprises one or more target loci comprising transplant-derived alleles; and wherein the determining step further comprises determining an amount of a transplant-derived allele at the one or more SNP loci, and determining whether the amount of the transplant-derived allele at the one or more SNP loci or a function thereof exceeds a cutoff threshold indicating transplant rejection; wherein transplant rejection is determined by a combination of (i) the amount of the transplant-derived allele at the one or more target loci or a function thereof, and (ii) the total amount of dd-cfDNA or the fraction of dd-cfDNA.

In one aspect, the present disclosure relates to a method of administrating immunosuppressive therapy in a maternal transplant recipient, comprising: (a) quantifying the total amount of cfDNA and the amount of dd-cfDNA in a biological sample of the transplant recipient according to the methods described herein; and (b) titrating the dosage of an immunosuppressive therapy according to the amount of cfDNA or a function thereof and the amount of dd-cfDNA or a function thereof. In some embodiments, the method further comprises repeating step (a) longitudinally for the same transplant recipient, and determining a longitudinal change in the amount of cfDNA or a function thereof and a longitudinal change in the amount of dd-cfDNA or a function thereof. In some embodiments, the method further comprises titrating the dosage of the immunosuppressive therapy according to the longitudinal change in the total amount of cfDNA or a function thereof and the longitudinal change in the amount of dd-cfDNA or a function thereof.

In some embodiments of the methods described herein, an increase in the levels of dd-cfDNA are indicative of transplant rejection and a need for adjusting immunosuppressive therapy. In some embodiments of the methods described herein, change or a decrease in the levels of dd-cfDNA indicates transplant tolerance or stability, and a need for adjusting immunosuppressive therapy.

In some embodiments of the methods described herein, the selected target loci comprise one or more single nucleotide polymorphisms (SNPs).

In some embodiments, the methods described herein are performed without prior knowledge of donor and/or recipient genotypes.

In some embodiments of the methods described herein, universal amplification of the extracted DNA is performed. In some embodiments, universal amplification preferentially amplifies dd-cfDNA over rd-cfDNA and fd-cfDNA.

In some embodiments of the methods described herein, extracting the cfDNA from the biological sample comprises size selection to enrich for dd-cfDNA and reduce the amount of rd-cfDNA and fd-cfDNA.

In some embodiments of the methods described herein, the amount of cfDNA is measured by quantitative PCR, real-time PCR, digital PCR, sequencing, microarray, or molecular barcodes and microscopic imaging (such as NanoString nCounter®). In some embodiments of the methods described herein, the amount of dd-cfDNA is determined by using ratiometric and/or machine learning-artificial intelligence comparisons at a single or a plurality of time points.

In some embodiments of the methods described herein, the cutoff threshold is an estimated percentage of dd-cfDNA out of total cfDNA or a function thereof. In some embodiments of the methods described herein, the amount of dd-cfDNA of greater than 1% of total cfDNA indicates that the transplant is undergoing acute rejection, and wherein an amount of dd-cfDNA of less than 1% of total cfDNA indicates that the transplant is either undergoing borderline rejection, undergoing other injury, or is stable.

In some embodiments of the methods described herein, the transplant recipient has received one or more transplants selected from kidney, liver, pancreas, intestinal, heart, lung, heart/lung, stomach, testis, penis, ovary, uterus, thymus, face, hand, leg, bone, bone marrow, cornea, skin, pancreas islet cell, heart valve, blood vessel, and blood transfusion. In some embodiments of the methods described herein, is obtained from the transplant recipient less than 18 months post-transplantation.

In some embodiments of the methods described herein, the rejection risk for the transplant recipient is determined using logistic regression, random forest, or decision tree machine learning analysis. In some embodiments of the methods described herein, the logistic regression, random forest, or decision tree machine learning analysis further incorporates one or more parameters selected from time post-transplantation, age of transplant recipient and/or transplant donor, gender of transplant recipient and/or transplant donor.

In some embodiments of the methods described herein, the biological sample is blood, serum, plasma, or urine.

### BRIEF DESCRIPTION OF THE DRAWINGS

FIG. 1. Workflow for detecting donor-derived cell-free DNA in a human plasma sample of a pregnant transplant recipient.

FIG. 2. Workflow for measuring dd-cfDNA fraction in pregnant kidney transplant recipients (KTRs).

FIG. 3. Vignettes representing dd-cfDNA testing and the medical history in two pregnant KTRs.

FIG. 4. Heterozygosity Rate Plots in two pregnant KTRs with (a) low and (b) high DFE.

FIG. 5. Measurements of donor-derived fractions across pregnant people with KTR.

### DETAILED DESCRIPTION

Sigdel et al., “Optimizing Detection of Kidney Transplant Injury by Assessment of Donor-Derived Cell-Free DNA via Massively Multiplex PCR,” J. Clin. Med. 8(1):19 (2019 ), is incorporated herein by reference in its entirety.

WO2020/010255, titled “Methods for Detection of Donor-Derived Cell-Free DNA” and filed on Jul. 3, 2019 as PCT/US2019/040603, is incorporated herein by reference in its entirety.

WO2021/243045, titled “Methods for Detection of Donor-Derived Cell-Free DNA” and filed on May 27, 2021 as PCT/US2021/034561, is incorporated herein by reference in its entirety.

The present disclosure relates to methods of determining and monitoring transplant rejection in a pregnant recipient based on targeted enrichment and high-throughput sequencing of cell-free DNA of a biological sample of the transplant recipient. In some embodiments, the cell-free DNA (cfDNA) is isolated from a biological sample, such as a blood, plasma, serum, or urine sample, of a transplant recipient. In some embodiments, the examples presented herein show that the presently disclosed methods can be used to detect nucleic acids derived from an donor in a biological sample taken from a pregnant transplant recipient.

### Methods of Determining and Monitoring Transplant Rejection Based on Measuring Cell-Free DNA.

In one aspect, the present disclosure relates to a method of preparing a composition of DNA derived from a biological sample of a maternal transplant recipient useful for determination of transplant status, comprising:
(a) extracting cell-free DNA (cfDNA) from the biological sample of the maternal transplant recipient, wherein the extracted cfDNA comprises donor-derived cell-free DNA (dd-cfDNA) from the transplant, recipient-derived cell-free DNA (rd-cfDNA) from the maternal transplant recipient, and fetal-derived cell-free DNA (fd-cfDNA) from a fetus;
(b) preparing a composition from the cfDNA wherein one or more selected target loci are enriched, wherein the maternal transplant recipient and biological father of the fetus are homozygous at the selected target loci to ensure homozygosity of the fetus at the selected target loci, such that heterozygosity observed in the extracted cfDNA at the selected target loci originates from the transplant; and
(c) quantifying the amount of extracted cfDNA and the amount of dd-cfDNA based on heterozygosity at the selected target loci to determine whether the amount of dd-cfDNA or a function thereof exceeds a cutoff threshold indicating transplant rejection. In some embodiments, the method further comprises preparing a sequencing library from the extracted cfDNA and sequencing the sequencing library by high-throughput sequencing to obtain sequencing reads. In some embodiments, no amplification or pre-amplification is performed on the extracted cfDNA prior to sequencing. In some embodiments, the selected target loci comprises 10-50,000 target loci, and the method further comprises performing multiplex targeted amplification of the DNA at the 10-50,000 target loci in a single reaction volume.

In one aspect, the present disclosure relates to a method of preparing a composition of DNA derived from a biological sample of a maternal transplant recipient useful for determination of transplant status, comprising:
(a) extracting cfDNA from the biological sample of the maternal transplant recipient, wherein the extracted cfDNA comprises donor-derived cell free DNA (dd-cfDNA), recipient-derived cell-free DNA (rd-cfDNA), and fetal-derived cell-free DNA (fd-cfDNA);
(b) preparing a sequencing library from the extracted cfDNA, sequencing the sequencing library by high-throughput sequencing to obtain sequencing reads, and quantifying the total amount of dd-cfDNA based on the sequencing reads;
(c) determining whether the amount of dd-cfDNA or a function thereof exceeds a cutoff threshold indicating transplant rejection or injury. In some embodiments, no amplification or pre-amplification is performed on the extracted cfDNA prior to sequencing. In some embodiments, multiplex targeted amplification is performed on the extracted cfDNA at 10-50,000 target loci in a single reaction volume, prior to sequencing. In some embodiments, preparing a sequencing library comprises attaching adapters to the extracted cfDNA for example by ligation. In some embodiments, attaching adapters to the extracted cfDNA comprises end repair, addition of an adenosine to the cfDNA fragments, followed by sticky end ligation to the cfDNA fragments. In some embodiments, the cfDNA fragments are repaired and filled to generated blunt end. In some embodiments, attaching adapters to the extracted cfDNA comprises blunt end ligation of adapters to the cfDNA fragments. In some embodiments, the adapters are attached during the amplification step.

In one aspect, the present disclosure relates to a method of preparing a composition of amplified DNA derived from a biological sample of a maternal transplant recipient useful for determination of transplant status, comprising:
(a) extracting cfDNA from the biological sample of the maternal transplant recipient, wherein the extracted cfDNA comprises dd-cfDNA from the transplant, rd-cfDNA, and fetal-derived cell free DNA;
(b) preparing a composition of amplified DNA by performing multiplex targeted amplification of the extracted cfDNA at 10-50,000 target loci in a single reaction volume to detect and quantify the amount of dd-cfDNA; and
(c) determining whether the amount of dd-cfDNA or a function thereof exceeds a cutoff threshold indicating transplant rejection.

In one aspect, the present disclosure relates to a method of preparing a composition of amplified DNA derived from a biological sample of a maternal transplant recipient useful for determination of transplant status, comprising:
(a) extracting cfDNA from the blood of the maternal transplant recipient, wherein the extracted cfDNA comprises dd-cfDNA, rd-cfDNA, and fd-cfDNA;
(b) preparing a composition of amplified DNA by performing targeted amplification of the extracted cfDNA at 10-50,000 target loci in a single reaction volume, and sequencing the amplified DNA by high-throughput sequencing to obtain sequencing reads and quantifying the amount of dd-cfDNA, transplant rd-cfDNA, and fd-cfDNA based on the sequencing reads, wherein the donor, recipient, and fetal loci are the same, and wherein the donor, recipient, and fetal reads are distinguished based on an insert sequence;
(c) determining whether the fraction of dd-cfDNA or a function thereof exceeds a cutoff threshold indicating transplant rejection. As used herein, “an insert sequence” refers to any sequence that is different in the target loci of the transplant recipient compared to the same target loci of the transplant donor.

In one aspect, the present disclosure relates to a method of preparing a composition of amplified DNA derived from a biological sample of a maternal transplant recipient useful for determination of transplant status, comprising:
(a) extracting cfDNA from the biological sample of the maternal transplant recipient, wherein the extracted cfDNA comprises dd-cfDNA, rd-cfDNA, and fd-cfDNA;
(b) preparing a composition of amplified DNA by performing targeted amplification of the extracted cfDNA at 10-50,000 target loci in a single reaction volume, wherein the target loci comprise one or more target loci indicating transplant rejection; and
(c) determining an amount of the one or more target loci indicating transplant rejection, and determining whether the amount of the one or more target loci indicating transplant rejection or a function thereof exceeds a cutoff threshold indicating transplant rejection.

In one aspect, the present disclosure relates to a method of preparing a composition of DNA derived from a biological sample of a maternal transplant recipient useful for determination of transplant status, wherein the composition of DNA comprises one or more target loci indicating transplant rejection; and wherein the determining step further comprises determining an amount of the one or more target loci indicating transplant rejection, and determining whether the amount of the one or more target loci indicating transplant rejection or a function thereof exceeds a cutoff threshold indicating transplant rejection; wherein transplant rejection is determined by a combination of (i) the amount of the one or more target loci indicating transplant rejection or a function thereof, and (ii) the total amount of dd-cfDNA or the fraction of dd-cfDNA.

In one aspect, the present disclosure relates to a method of administrating immunosuppressive therapy in a maternal transplant recipient, comprising:
(a) quantifying the total amount of cfDNA and the amount of dd-cfDNA in a biological sample of the transplant recipient according to any of the methods disclosed herein; and
(b) titrating the dosage of an immunosuppressive therapy according to the amount of cfDNA or a function thereof and the amount of dd-cfDNA or a function thereof. In some embodiments, the method further comprises repeating step (a) longitudinally for the same transplant recipient, and determining a longitudinal change in the amount of cfDNA or a function thereof and a longitudinal change in the amount of dd-cfDNA or a function thereof.

In some embodiments, the cfDNA is derived from extracellular vesicles (EVs) isolated from a biological sample such as blood, plasma, serum or urine samples of a maternal transplant recipient.

### Samples Comprising Nucleic Acids and Methods for Obtaining Samples and Extracting Nucleic Acids

The methods disclosed herein comprises extracting fragmented or intact cfDNA derived from a sample obtained from a maternal transplant recipient. In some embodiments, the transplant recipient is a human subject, and the transplant donor is a human. In some embodiments, the transplant is from a pig, a primate, a baboon, a cow, or a dog. In some embodiments, the transplant can be an allograft or a xenograft. In some embodiments, the transplant can be an organ transplant, tissue transplant, cell transplant, or fluid transplant.

In some embodiments, the transplant recipient has received a plurality of transplanted organs selected from kidney, liver, pancreas, intestinal, heart, lung, heart/lung, stomach, testis, penis, ovary, uterus, thymus, face, hand, leg, bone, bone marrow, cornea, skin, pancreas islet cell, heart valve, and blood vessel. In some embodiments, the one or more transplanted organs are from the same transplant donors. In some embodiments, the one or more transplanted organs are from more multiple different transplant donors. In some embodiments, the transplant recipient has received simultaneous transplantation of more than one organ. In some embodiments the transplant recipient has received a blood transfusion. In some embodiments the blood transfusion is from the same donor as one or more of the organ transplants. In some embodiments the blood transfusion is from a different donor to the organ donor.

In some embodiments, the transplant recipient has received one or more transplanted organs selected from kidney, liver, heart, lung, pancreas, intestinal, thymus, and uterus. In some embodiments, the transplant recipient has received a kidney transplant. In some embodiments, the transplant recipient has received a liver transplant. In some embodiments, the transplant recipient has received a heart transplant. In some embodiments, the transplant recipient has received a lung transplant. In some embodiments, the transplant recipient has received a pancreas transplant. In some embodiments, the transplant recipient has received an intestinal transplant. In some embodiments, the transplant recipient has received a thymus transplant. In some embodiments, the transplant recipient has received a uterus transplant.

In some embodiments, the sample is obtained from the transplant recipient less than 18 months post-transplantation, less than 17 months post-transplantation, less than 16 months post-transplantation, less than 15 months post-transplantation, less than 14 months post-transplantation, less than 13 months post-transplantation, or less than 12 months post-transplantation. In some embodiments, the sample is obtained from the transplant recipient between 0 and 2 months post-transplantation, between 2 and 4 months post-transplantation, between 4 and 6 months post-transplantation, between 6 and 9 months post-transplantation, between 9 and 12 months post-transplantation, or between 12 and 18 months post-transplantation.

In some embodiments, the transplant recipient is pregnant. In some embodiments the transplant recipient is in the first trimester of pregnancy. In some embodiments, the transplant recipient is in the second trimester of pregnancy. In some embodiments, the transplant recipient is in the third trimester of pregnancy. In some embodiments, the transplant recipient is less than 3 months pregnant. In some embodiments, the transplant recipient is less than six months pregnant. In some embodiments, the transplant recipient is less than nine months pregnant. In some embodiments, the transplant recipient is more than nine months pregnant. In some embodiments, the transplant recipient has recently given birth. In some embodiments, the transplant recipient has given birth less than one day ago, i.e. is less than one day postpartum. In some embodiments, the transplant recipient has given birth less than one week ago, i.e. is less than one week postpartum. In some embodiments, the transplant recipient has given birth less than one month ago, i.e. is less than one month postpartum. In some embodiments, the transplant recipient has given birth less than three months ago, i.e. is less than three months postpartum. In some embodiments, the transplant recipient has given birth less than one year ago, i.e. is less than one year postpartum. In some embodiments, there is a single fetus. In some embodiments there are multiple fetus'. In some embodiments, the transplant recipient is not pregnant.

In some embodiments, the methods disclosed herein further comprise measuring the amounts of total cfDNA and dd-cfDNA longitudinally for the same transplant recipient and determining a longitudinal change in the amount of total cfDNA and dd-cfDNA. In some embodiments, the amounts of dd-cfDNA is the total amount of cfDNA derived from the donor organ.

In some embodiments, the transplant recipient has received one or more organs from the same transplant donor. In some embodiments, the transplant recipient has received one or more organs from multiple different transplant donors. In some embodiments, the transplant recipient has received simultaneous transplantation of more than one organ from one or more different donors.

The biological samples may be a body fluid sample, a tissue, an organ, or individual cells. In some embodiments the biological sample comprises blood, plasma, serum, CSF, or urine. In some embodiments, the biological sample is blood. In some embodiments, the sample is blood, plasma, or serum. In some embodiments, the biological samples may be extracellular vehicles derived from the body fluid samples such as blood, plasma, serum, CSF, or urine.

### Nucleic Acids and Methods of Extracting or Enriching Nucleic Acids

The methods disclosed herein comprises extracting nucleic acids from a sample derived from a subject. In some embodiments, the subject may be the pregnant transplant recipient. In some embodiments, the subject may be the father of the transplant recipient's fetus. In some embodiments the subject may be the transplant donor. In some embodiments, the subject may be a fetus. The nucleic acids may be genomic DNA, cDNA, cell-free DNA (cfDNA), cell-free mitochondrial DNA (cf mDNA), cell-free DNA that originated from nuclear DNA (cf nDNA), cellular DNA, or mitochondrial DNA. The cfDNA may be derived from exosomes or microvesicles. In some embodiments, the nucleic acids may be RNA such as cell-free RNA, cellular RNA, or RNA extracted from exosomes. The term “RNA” refers herein to any type of RNA, including messenger RNA (mRNA) or small non-coding RNA (sncRNA) such as micro RNA (miRNA), or a mixture thereof. In some embodiments, the RNA may be cell-free, cellular, or exosome RNA. In some embodiments, the RNA comprises small non-coding RNA (sncRNA). In some embodiments, the sncRNA comprises micro RNA (miRNA), piwi-interacting RNA (piRNA), small nucleolar RNA (snoRNA), small nuclear RNA (snRNA), or miscellaneous RNA (miscRNA). In some embodiments, the cell-free sncRNA is derived from exosomes or microvesicles.

In some embodiments, nucleic acids are extracted by using size exclusion. In some embodiments, cfDNA is isolated from cellular DNA based on size. In some embodiments, nucleic acids are isolated by using magnetically or otherwise labelled affinity chromatography.

In some embodiments, nucleic acids are preferentially enriched. Nucleic acids may be preferentially enriched by using preferential enrichment at a locus or target site. Such preferential enrichment refers to any method that results in the percentage of molecules of nucleic acids in a post-enrichment nucleic acid mixture that correspond to the locus being higher than the percentage of molecules of nucleic acids in the pre-enrichment nucleic acid mixture that correspond to the locus. The method may involve selective amplification of nucleic acid molecules that correspond to a locus. The method may involve removing nucleic acid molecules that do not correspond to the locus. The method may involve a combination of methods. The degree of enrichment is defined as the percentage of molecules of nucleic acids in the post-enrichment mixture that correspond to the locus or target divided by the percentage of molecules of nucleic acids in the pre-enrichment mixture that correspond to the locus or target. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

Amplification refers to a method that increases the number of copies of nucleic acid molecules. Selective amplification may refer to a method that increases the number of copies of a particular nucleic acid molecules, or nucleic acid molecules that correspond to a particular region of nucleic acid molecules. It may also refer to a method that increases the number of copies of a particular targeted molecule of nucleic acid molecules, or targeted region of nucleic acid molecules more than it increases non-targeted molecules or regions of nucleic acid molecules.

Selective amplification may be a method of preferential enrichment. Universal Priming Sequence refers to a DNA sequence that may be appended to a population of target DNA molecules, for example by ligation, PCR, or ligation mediated PCR. Once added to the population of target molecules, primers specific to the universal priming sequences can be used to amplify the target population using a single pair of amplification primers. Universal priming sequences are typically not related to the target sequences. Universal Adapters, or ligation adaptors or library tags are DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers. Targeting refers to a method used to selectively amplify or otherwise preferentially enrich those molecules of DNA that correspond to a set of loci, in a mixture of DNA.

Particular nucleic acids may also be enriched for by using hybrid capture. In some embodiments, preferentially enriching the DNA at the plurality of loci or target sites comprises: obtaining a set of hybrid capture probes; hybridizing the hybrid capture probes to the DNA in the sample; and physically separating the hybridized DNA from the sample of DNA from the unhybridized DNA from the sample.

In some embodiments, in the methods disclosed herein, the DNA is preferentially enriched at the target loci or a biomarker.

The term “biomarker” refers to a molecule that is an indicator of an abnormal biological condition (e.g., disease or disorder, or transplant rejection). For example, a biomarker may be a gene or gene product (i.e. RNA or protein) that (a) is expressed at higher or lower levels, (b) has an altered ratio relative to another biomarker, (c) is present at higher or lower levels, (d) is a variant or mutant of the gene product, or (e) is simply present or absent, in a cell or tissue sample from a subject having or suspected of having a disease as compared to an undiseased tissue or cell sample from the subject having or suspected of having a disease, or as compared to a cell or tissue sample from a subject or a pool of subjects not having or suspected of having the disease. In the context of transplant, the biomarker may be indicative of poor donor organ health or transplant rejection. That is, one or more gene products are sufficiently specific to the test sample that one or more may be used to identify, predict, or detect the presence of transplant rejection, disease, risk of disease, risk of a given event or change in disease status, or provide information for a proper or improved therapeutic regimen. In the methods described herein, levels of dd-cfDNA above a threshold may be considered a biomarker.

In some embodiments, one or more biomarkers can be one or a collection of genetic aberrations, which is used herein to refer to the nucleic acid amounts as well as nucleic acid variants within the nucleic acid-containing particles. Specifically, genetic aberrations include, without limitation, over-expression of a gene (e.g., an oncogene) or a panel of genes, under-expression of a gene (e.g., a tumor suppressor gene such as p53 or RB) or a panel of genes, alternative production of splice variants of a gene or a panel of genes, gene copy number variants (CNV) (e.g., DNA double minutes), nucleic acid modifications (e.g., methylation, acetylation and phosphorylations), single nucleotide polymorphisms (SNPs), chromosomal rearrangements (e.g., inversions, deletions and duplications), and mutations (insertions, deletions, duplications, missense, nonsense, synonymous or any other nucleotide changes) of a gene or a panel of genes, which mutations, in many cases, ultimately affect the activity and function of the gene products, lead to alternative transcriptional splice variants and/or changes of gene expression level, or combinations of any of the foregoing.

In some embodiments, preferentially enriching the DNA in the sample at the plurality of polymorphic loci includes obtaining a plurality of pre-circularized probes where each probe targets one of the polymorphic loci, and where the 3′ and 5′ end of the probes are designed to hybridize to a region of DNA that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the pre-circularized probes to DNA from the sample, filling the gap between the hybridized probe ends using DNA polymerase, circularizing the pre-circularized probe, and amplifying the circularized probe.

In some embodiments, preferentially enriching the DNA at the plurality of polymorphic loci includes obtaining a plurality of ligation-mediated PCR probes where each PCR probe targets one of the polymorphic loci, and where the upstream and downstream PCR probes are designed to hybridize to a region of DNA, on one strand of DNA, that is separated from the polymorphic site of the locus by a small number of bases, where the small number is 1, 2, 3, 4, 5, 6, 7, 8, 9, 10, 11, 12, 13, 14, 15, 16, 17, 18, 19, 20, 21 to 25, 26 to 30, 31 to 60, or a combination thereof, hybridizing the ligation-mediated PCR probes to the DNA from the first sample, filling the gap between the ligation-mediated PCR probe ends using DNA polymerase, ligating the ligation-mediated PCR probes, and amplifying the ligated ligation-mediated PCR probes.

In some embodiments, preferentially enriching the DNA at the plurality of polymorphic loci includes obtaining a plurality of hybrid capture probes that target the polymorphic loci, hybridizing the hybrid capture probes to the DNA in the sample and physically removing some or all of the unhybridized DNA from the first sample of DNA.

In some embodiments, the hybrid capture probes are designed to hybridize to a region that is flanking but not overlapping the polymorphic site. In some embodiments, the hybrid capture probes are designed to hybridize to a region that is flanking but not overlapping the polymorphic site, and where the length of the flanking capture probe may be selected from the group consisting of less than about 120 bases, less than about 110 bases, less than about 100 bases, less than about 90 bases, less than about 80 bases, less than about 70 bases, less than about 60 bases, less than about 50 bases, less than about 40 bases, less than about 30 bases, and less than about 25 bases. In some embodiments, the hybrid capture probes are designed to hybridize to a region that overlaps the polymorphic site, and where the plurality of hybrid capture probes comprise at least two hybrid capture probes for each polymorphic loci, and where each hybrid capture probe is designed to be complementary to a different allele at that polymorphic locus.

In some embodiments, preferentially enriching the DNA at a plurality of polymorphic loci includes obtaining a plurality of inner forward primers where each primer targets one of the polymorphic loci, and where the 3′ end of the inner forward primers are designed to hybridize to a region of DNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, optionally obtaining a plurality of inner reverse primers where each primer targets one of the polymorphic loci, and where the 3′ end of the inner reverse primers are designed to hybridize to a region of DNA upstream from the polymorphic site, and separated from the polymorphic site by a small number of bases, where the small number is selected from the group consisting of 1, 2, 3, 4, 5, 6 to 10, 11 to 15, 16 to 20, 21 to 25, 26 to 30, or 31 to 60 base pairs, hybridizing the inner primers to the DNA, and amplifying the DNA using the polymerase chain reaction to form amplicons.

In some embodiments, the method also includes obtaining a plurality of outer forward primers where each primer targets one of the polymorphic loci, and where the outer forward primers are designed to hybridize to the region of DNA upstream from the inner forward primer, optionally obtaining a plurality of outer reverse primers where each primer targets one of the polymorphic loci, and where the outer reverse primers are designed to hybridize to the region of DNA immediately downstream from the inner reverse primer, hybridizing the first primers to the DNA, and amplifying the DNA using the polymerase chain reaction.

In some embodiments, the method also includes obtaining a plurality of outer reverse primers where each primer targets one of the polymorphic loci, and where the outer reverse primers are designed to hybridize to the region of DNA immediately downstream from the inner reverse primer, optionally obtaining a plurality of outer forward primers where each primer targets one of the polymorphic loci, and where the outer forward primers are designed to hybridize to the region of DNA upstream from the inner forward primer, hybridizing the first primers to the DNA, and amplifying the DNA using the polymerase chain reaction.

In some embodiments, preparing the sample comprises incorporating universal adapters in to the DNA and amplifying the DNA using the polymerase chain reaction. In some embodiments, at least a fraction of the amplicons that are amplified are less than 100 bp, less than 90 bp, less than 80 bp, less than 70 bp, less than 65 bp, less than 60 bp, less than 55 bp, less than 50 bp, or less than 45 bp, and where the fraction is 10%, 20%, 30%, 40%, 50%, 60%, 70%, 80%, 90%, or 99%.

In some embodiments, amplifying the DNA is done in one or a plurality of individual reaction volumes, and where each individual reaction volume contains more than 10 different forward and reverse primer pairs, more than 100 different forward and reverse primer pairs, more than 200 different forward and reverse primer pairs, more than 500 different forward and reverse primer pairs, more than 1,000 different forward and reverse primer pairs, more than 2,000 different forward and reverse primer pairs, more than 5,000 different forward and reverse primer pairs, more than 10,000 different forward and reverse primer pairs, more than 20,000 different forward and reverse primer pairs, more than 50,000 different forward and reverse primer pairs, or more than 100,000 different forward and reverse primer pairs.

In some embodiments, preparing the sample further comprises dividing the sample into a plurality of portions, and where the DNA in each portion is preferentially enriched at a subset of the plurality of polymorphic loci. In some embodiments, the inner primers are selected by identifying primer pairs likely to form undesired primer duplexes and removing from the plurality of primers at least one of the pair of primers identified as being likely to form undesired primer duplexes. In some embodiments, the inner primers contain a region that is designed to hybridize either upstream or downstream of the targeted polymorphic locus, and optionally contain a universal priming sequence designed to allow PCR amplification. In some embodiments, at least some of the primers additionally contain a random region that differs for each individual primer molecule. In some embodiments, at least some of the primers additionally contain a molecular barcode.

In some embodiments, the method comprises: (a) performing multiplex polymerase chain reaction (PCR) on a nucleic acid sample comprising target loci to simultaneously amplify at least 10 distinct target loci using either (i) at least 10 different primer pairs, or (ii) at least 10 target-specific primers and a universal or tag-specific primer, in a single reaction volume to produce amplified products comprising target amplicons; and (b) sequencing the amplified products. In some embodiments, the method does not comprise using a microarray.

In some embodiments, the method comprises (a) performing multiplex polymerase chain reaction (PCR) on the cfDNA sample comprising target loci to simultaneously amplify at least 10 distinct target loci using either (i) at least 10 different primer pairs, or (ii) at least 10 target-specific primers and a universal or tag-specific primer, in a single reaction volume to produce amplified products comprising target amplicons; and b) sequencing the amplified products. In some embodiments, the method does not comprise using a microarray.

After blood draw and before nucleic acid extraction, blood cells within a blood sample may burse and shed long fragments of DNA into the sample, which would increase the total amount of cfDNA and background noise, distorting the % of dd-cfDNA detected. In order to reduce such background noise, and based on the observation that dd-cfDNA is typically shorter than DNA shredded from a transplant recipient blood cell, two particular enrichments for dd-cfDNA are contemplated. In one embodiment, a size selection is applied to select for shorter cfDNA. In another embodiment, a universal amplification step is applied to reduce noise (e.g., before applying multiplex PCR), based on the hypothesis that shorter dd-cfDNA (often in mononucleosome form) is amplified more efficiently than longer transplant recipient-derived DNA.

### Target Genes and Loci

In some embodiments, the methods disclosed herein comprise selecting target loci that are homozygous in both the maternal transplant recipient and in the biological father of the fetus, ensuring homozygosity in the fetus. Any heterozygosity at the loci detected in the extracted cfDNA will therefore be from the donor. In some embodiments, heterozygosity detected in the extracted cfDNA can be used to quantify the amount of dd-cfDNA present in the biological sample.

In some embodiments, the methods disclosed herein further comprise preferentially enriching the cfDNA at a plurality of target loci selected based on homozygosity in both the maternal transplant recipient and biological father of the fetus. In some embodiments, between 10 and 50,000 target loci are selected and enriched.

In some embodiments, the methods described herein further comprise sequencing the paternal genome of the fetus in order to select the target loci at which both the maternal transplant recipient and biological father of the fetus are homozygous. In some embodiments, the paternal genotype of the fetus is already known.

The nucleic acids may comprise biomarkers indicative of an immune response, or various diseases or conditions as described elsewhere herein. In some embodiments, the target loci comprise one or more different sets of target loci. In some embodiments, the target loci comprises a set of recipient target loci, a set of fetal target loci, and a set of donor target loci, wherein each of the sets of recipient, fetal, and donor target loci are different. In some embodiments, the sets of recipient and fetal target loci are the same, and the set of donor target loci are different. In some embodiments, each of the sets of recipient, fetal, and donor target loci are all the same, and one or more recipient and/or fetal target loci can be distinguished from the corresponding donor loci by an insert sequence. As used herein, “an insert sequence” refers to any sequence that is different in the target loci of the transplant recipient and/or fetus compared to the same target loci of the transplant donor.

In some embodiments, the method comprises extracting fragmented or intact cfDNA derived from sample of the transplant recipient, wherein the extracted cfDNA comprises donor-, fetal-, and/or recipient-derived cfDNA, and wherein the cfDNA comprises a plurality of biomarkers indicative of an immune response, or a disease or disorder. In some embodiments, the biomarker indicates an increased immune response. In some embodiments, the biomarker indicates a decreased immune response.

In some embodiments, the presently disclosed method comprises pre-selecting cfDNA target molecules. In some embodiments, the cfDNA target molecules comprise cfDNA species known to be relevant for assessing organ health. In some embodiments, the present disclosure provides methods for identifying cfDNA target molecules that are relevant for assessing organ health.

In some embodiments, the methods disclosed herein further comprise preferentially enriching the cfDNA at a plurality of target loci or biomarkers indicative of transplant rejection. In some embodiments, the cfDNA biomarkers indicate an increased immune response, or a decreased immune response.

In some embodiments, the target loci and/or biomarkers comprise single nucleotide polymorphism (SNP) loci.

Samples and Methods for Isolating Nucleic Acids from the Samples

In some embodiments, the nucleic acid sample includes fragmented or digested nucleic acids. In some embodiments, the nucleic acid sample includes DNA, such as genomic DNA, cDNA, cell-free DNA (cfDNA), cell-free mitochondrial DNA (cf mDNA), cell-free DNA that originated from nuclear DNA (cf nDNA), cellular DNA, or mitochondrial DNA.

In some embodiments, the nucleic acid sample includes DNA from a single cell, 2 cells, 3 cells, 4 cells, 5 cells, 6 cells, 7 cells, 8 cells, 9 cell, 10 cells, or more than 10 cells. In some embodiments, the nucleic acid sample is a blood or plasma sample that is substantially free of cells. In some embodiments, the nucleic acid sample includes or is derived from blood, plasma, saliva, semen, sperm, cell culture supernatant, mucus secretion, dental plaque, gastrointestinal tract tissue, stool, urine, hair, bone, body fluids, tears, tissue, skin, fingernails, blastomeres, embryos, amniotic fluid, chorionic villus samples, bile, lymph, cervical mucus, or a forensic sample. In some embodiments, the target loci are segments of nucleic acids. In some embodiments, the target loci are segments of nucleic acids found in the genome. In some embodiments, the target loci comprise or consist of single nucleotide polymorphisms (SNPs). In some embodiments, the primers are DNA molecules.

In some embodiments, the method includes isolating or purifying the DNA. There are a number of standard procedures known in the art to accomplish such an end. In some embodiments, the sample may be centrifuged to separate various layers. In some embodiments, the DNA may be isolated using filtration. In some embodiments, the preparation of the DNA may involve amplification, separation, purification by chromatography, liquid separation, isolation, preferential enrichment, preferential amplification, targeted amplification, or any of a number of other techniques either known in the art or described herein. In some embodiments for the isolation of DNA, RNase is used to degrade RNA. In some embodiments, an QIAamp™ DNA Mini Kit (Qiagen), is used to isolate DNA according to the manufacturer's protocol. In some embodiments, cfDNA molecules are isolated using MagMAX™ cell-free DNA isolation kit (Applied Biosystems). The concentration and purity of DNA may optionally be determined using Nanovue (GE Healthcare, Piscataway, N.J., USA), and DNA integrity may optionally be measured by use of the 2100 Bioanalyzer (Agilent Technologies, Santa Clara, Calif., USA).

In some embodiments, adaptors are added to make a sequencing library. Prior to ligation, sample DNA may be blunt ended, and then a single adenosine base is added to the 3-prime end. In some embodiments, ligation of adaptors to nucleic acids is a sticky end ligation. Prior to ligation the DNA may be cleaved using a restriction enzyme or some other cleavage method. During ligation the 3-prime adenosine of the sample fragments and the complementary 3-prime tyrosine overhang of adaptor can enhance ligation efficiency. In some embodiments, adaptor ligation is performed using the ligation kit found in the AGILENT SURESELECT™ kit. In some embodiments, the adapters are incorporated into the sequences by PCR. In some embodiments, the adapters are incorporated into the sequences during universal or targeted amplification.

In some embodiments, the library is amplified using universal primers. In an embodiment, the amplified library is fractionated by size separation or by using products such as AGENCOURT AMPURE™ beads or other similar methods. In some embodiments, PCR amplification is used to amplify target loci. In some embodiments, the amplified DNA is sequenced (such as sequencing using an ILLUMINA IIGAX™ or HiSeq sequencer). In some embodiments, the amplified DNA is sequenced from each end of the amplified DNA to reduce sequencing errors. If there is a sequence error in a particular base when sequencing from one end of the amplified DNA, there is less likely to be a sequence error in the complementary base when sequencing from the other side of the amplified DNA (compared to sequencing multiple times from the same end of the amplified DNA).

As non-limiting examples, a locus can be a single nucleotide polymorphism (SNP), an intron, or an exon. In some embodiments, a locus can include an insertion, deletion, or transposition. In some embodiments, the sample can include a blood, sera, or plasma sample. In some embodiments, the sample can include free floating DNA (e.g. circulating cell-free tumor DNA, circulating cell-free donor DNA, or circulating cell-free fetal DNA) in a blood, sera, or plasma sample. In these embodiments, the sample is typically from an animal, such as a mammal or human, and is typically present in fragments about 160 nucleotides in length. In some embodiments, the free-floating DNA is isolated from blood using an EDTA-2Na tube after removal of cellular debris and platelets by centrifugation. The plasma samples can be stored at 80 °C until the DNA is extracted using, for example, QIAamp™ DNA Mini Kit (Qiagen, Hilden, Germany), (e.g. Hamakawa et al., Br J Cancer. 2015; 112:352-356).

Many kits and methods are known in the art for generating libraries of nucleic acid molecules for subsequent sequencing. Kits especially adapted for preparing libraries from small nucleic acid fragments, especially circulating cell-free DNA, can be useful for practicing methods provided herein. For example, the NEXTflex™ Cell Free kits (Bioo Scientific, Austin, Tex.) or the Natera Library Prep Kit (Natera, San Carlos, Calif.). Such kits would typically be modified to include adaptors that are customized for the amplification and sequencing steps of the methods provided herein. Adaptor ligation can also be performed using commercially available kits such as the ligation kit found in the Agilent SureSelect™ kit (Agilent, Santa Clara, Calif.).

Sample nucleic acid molecules are composed of naturally occurring or non-naturally occurring ribonucleotides or deoxyribonucleotides linked through phosphodiester linkages.

Furthermore, sample nucleic acid molecules are composed of a nucleic acid segment that is targeted for sequencing. Sample nucleic acid molecules can be or can include nucleic acid segments that are at least 20, 25, 50, 75, 100, 125, 150, 200, 250, 300, 400, 500, 600, 700, 800, 900, or 1,000 nucleotides in length. In any of the embodiments disclosed herein the sample nucleic acid molecules or nucleic acid segments can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. In some embodiments, the nucleic acid molecules can be fragments of genomic DNA and can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. For the sake of clarity, nucleic acids initially isolated from a living tissue, fluid, or cultured cells, can be much longer than sample nucleic acid molecules processed using methods herein. As discussed herein, for example, such initially isolated nucleic acid molecules can be fragmented to generate nucleic acid segments, before being used in the methods herein. In some embodiments, the nucleic acid molecules and nucleic acid segments can be identical. The sample nucleic acid molecule or sample nucleic acid segment can include a target locus that contains the nucleotide or nucleotides that are being queried, especially a single nucleotide polymorphism or single nucleotide variant. In any of the disclosed embodiments, the target loci can be at least 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, 500, 600, 700, 800, 900, or 1,000 nucleotides in length and include a portion of or the entirety of the sample nucleic acid molecule and/or the sample nucleic acid segment. In other embodiments, the target loci can be between 1, 2, 3, 4, 5, 6, 7, 8, 9, 10, 11, 12, 13, 14, 15, 16, 17, 18, 19, 20, 25, 50, 75, 100, 125, 150, 200, 250, 300, 400, and 500 nucleotides in length on the low end of the range and 10, 11, 12, 13, 14, 15, 16, 17, 18, 19, 20, 25, 50, 75, 100, 125, 150, 200, 250, 300, 400, 500, 1,000, 2,000, 3,000, 4,000, 5,000, 6,000, 7,000, 8,000, 9,000, and 10,000 nucleotides in length on the high end of the range. In some embodiments, the target loci on different sample nucleic acid molecules can be at least 50%, 60%, 70%, 80%, 90% 95%, 96%, 97%, 98%, 99%, 99.9%, or 100% identical. In some embodiments, the target loci on different sample nucleic acid molecules can share at least 50%, 60%, 70%, 80%, 90% 95%, 96%, 97%, 98%, 99%, 99.9%, or 100% sequence identity.

In some embodiments, the entire sample nucleic acid molecule is a sample nucleic acid segment. For example, in certain embodiments where adaptors are ligated directly to the ends of sample nucleic acid molecules, or ligated to a nucleic acid(s) ligated to the ends of sample nucleic acid molecules, or ligated as part of primers that bind to sequences at the termini of sample nucleic acid segments, or adapters, such as universal adapters added thereto, as discussed further herein, the entire nucleic acid molecule can be a sample nucleic acid segment. In other embodiments, for example certain embodiments where adaptors are attached to sample nucleic acid molecules as part of primers that target binding sites internal to the termini of sample nucleic acid molecules, a portion of the sample nucleic acid molecule can be the sample nucleic acid segment that is targeted for downstream sequencing. For example, at least 50%, 60%, 70%, 75%, 80%, 85%, 90%, 95%, 96%, 97%, 98%, 99%, or 100% of a sample nucleic acid molecule can be a nucleic acid segment.

In some embodiments, sample nucleic acid molecules are a mixture of nucleic acids isolated from a natural source, some sample nucleic acid molecules having identical sequences, some having sequences sharing at least 50%, 60%, 70%, 80%, 90%, 95%, 98%, or 99% sequence identity, and some with less than 50%, 40%, 30%, 20%, 10%, or 5% sequence identity over between 20, 25, 50, 75, 100, 125, 150, 200, 250 nucleotides on the low end of the range, and 50, 75, 100, 125, 150, 200, 250, 300, 400, or 500 nucleotides on the high end of the range. Such sample nucleic acid molecules can be nucleic acid samples isolated from tissues or fluids of a mammal, such as a human, without enriching one sequence over another. In other embodiments, target sequences, for example, those from a gene of interest, can be enriched prior to performing methods provided herein.

Distinguishing dd-cfDNA from fd-cfDNA

In some embodiments, dd-cfDNA is distinguished from fd-cfDNA based on parental genotype. For example, in some embodiments, the paternal DNA from the biological father of the fetus is genotyped using methods described herein. In some embodiments, the paternal genotype is already known. Based on the paternal and maternal transplant recipient genotype, target loci are selected that are homozygous in both the maternal and paternal genomes, and therefore will be homozygous in the fetal genome. Heterozygosity detected in the cfDNA at the target loci will therefore originate from the transplant and can be used to quantify the amount of dd-cfDNA in the biological sample.

### Determination of Rejection Risk for the Transplant Recipient

In some embodiments, the rejection risk for the transplant recipient is determined using logistic regression, random forest, or decision tree machine learning analysis. In some embodiments, the machine learning analysis incorporates the amount of dd-cfDNA in the sample of the transplant recipient or a function thereof as a parameter. In some embodiments, the machine learning analysis incorporates the number of reads of dd-cfDNA or a function thereof as a parameter. In some embodiments, the machine learning analysis incorporates the estimated percentage of dd-cfDNA out of total cfDNA as a parameter. In some embodiments, the machine learning analysis incorporates the amount of dd-cfDNA, the number of reads of dd-cfDNA, or the estimated percentage of dd-cfDNA out of total cfDNA in the sample of the transplant recipient as a parameter.

Machine learning may be used to resolve rejection vs non-rejection. Machine learning is disclosed in WO2020/018522, titled “Methods and Systems for calling Ploidy States using a Neural Network” and filed on Jul. 16, 2019 as PCT/US2019/041981, which is incorporated herein by reference in its entirety. In some embodiments, the cutoff threshold value is scaled according to the amount of total cfDNA in the biological sample.

In some embodiments, the cutoff threshold value is expressed as percentage of dd-cfDNA (dd-cfDNA %) in the sample. In some embodiments, the cutoff threshold value is expressed as quantity or absolute quantity of dd-cfDNA. In some embodiments, the cutoff threshold value is expressed as quantity or absolute quantity of dd-cfDNA per volume unit of the blood sample. In some embodiments, the cutoff threshold value is expressed as quantity or absolute quantity of dd-cfDNA per volume unit of the blood sample multiplied by body mass, BMI, or blood volume of the transplant recipient.

In some embodiments, the cutoff threshold value takes into account the body mass, BMI, or blood volume of the patient. In some embodiments, the cutoff threshold value takes into account one or more of the following: donor genome copies per volume of plasma, fetal genome copies per volume of plasma, number of fetus' present, stage of pregnancy, cfDNA yield per volume of plasma, donor height, donor weight, donor age, donor gender, donor ethnicity, donor organ mass, donor organ, live vs deceased donor, the donor's familial relationship to the recipient (or lack thereof), recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient, fetal, and/or donor HLA alleles or epitope mismatches, Banff classification of renal transplant pathology, and for-cause vs surveillance or protocol biopsy.

In some embodiments, the method has a specificity of at least 50% in identifying acute rejection (AR) over non-AR when the dd-cfDNA amount is above the cutoff threshold value scaled or adjusted according to the amount of total cfDNA in the biological sample and a confidence interval of 95%. In some embodiments, the method has a specificity of at least 60% in identifying acute rejection (AR) over non-AR when the dd-cfDNA amount is above the cutoff threshold value scaled or adjusted according to the amount of total cfDNA in the biological sample and a confidence interval of 95%. In some embodiments, the method has a specificity of at least 70% in identifying acute rejection (AR) over non-AR when the dd-cfDNA amount is above the cutoff threshold value scaled or adjusted according to the amount of total cfDNA in the biological sample and a confidence interval of 95%. In some embodiments, the method has a specificity of at least 75% in identifying acute rejection (AR) over non-AR when the dd-cfDNA amount is above the cutoff threshold value scaled or adjusted according to the amount of total cfDNA in the biological sample and a confidence interval of 95%. In some embodiments, the method has a specificity of at least 80% in identifying acute rejection (AR) over non-AR when the dd-cfDNA amount is above the cutoff threshold value scaled or adjusted according to the amount of total cfDNA in the biological sample and a confidence interval of 95%. In some embodiments, the method has a specificity of at least 85% in identifying acute rejection (AR) over non-AR when the dd-cfDNA amount is above the cutoff threshold value scaled or adjusted according to the amount of total cfDNA in the biological sample and a confidence interval of 95%. In some embodiments, the method has a specificity of at least 90% in identifying acute rejection (AR) over non-AR when the dd-cfDNA amount is above the cutoff threshold value scaled or adjusted according to the amount of total cfDNA in the biological sample and a confidence interval of 95%. In some embodiments, the method has a specificity of at least 95% in identifying acute rejection (AR) over non-AR when the dd-cfDNA amount is above the cutoff threshold value scaled or adjusted according to the amount of total cfDNA in the biological sample and a confidence interval of 95%.

Some embodiments use either a fixed threshold of dd-cfDNA per plasma volume or one that is not fixed, such as adjusted or scaled as noted herein. The way that this is determined can be based on using a training data set to build an algorithm to maximize performance. It may also take into account other data such as patient weight, age, or other clinical factors described herein.

In some embodiments, the method further comprises determining the occurrence or likely occurrence of transplant rejection using the amount of dd-cfDNA. In some embodiments, the amount of dd-cfDNA is compared to a cutoff threshold value to determine the occurrence or likely occurrence of transplant rejection, wherein the cutoff threshold value is adjusted or scaled according to the amount of total cfDNA. In some embodiments, the cutoff threshold value is a function of the number of reads of the dd-cfDNA.

In some embodiments, the method comprises applying a scaled or dynamic threshold metric that takes into account the amount of total cfDNA in the samples to more accurately assess transplant rejection. In some embodiments, the method further comprises flagging the sample if the amount of total cfDNA is above a pre-determined value. In some embodiments, the method further comprises flagging the sample if the amount of total cfDNA is below a pre-determined value.

In some embodiments, the machine learning analysis further incorporates time post-transplantation as a parameter. In some embodiments, the machine learning analysis further incorporates the age of transplant recipient and/or transplant donor as a parameter. In some embodiments, the machine learning analysis further incorporates the stage of the pregnancy as a parameter. In some embodiments, the machine learning analysis further incorporates the number of fetus' as a parameter. In some embodiments, the machine learning analysis further incorporates the gender of transplant recipient, the gender of the fetus(s), and/or the gender of the transplant donor as a parameter.

In some embodiments, the rejection risk for the transplant recipient is determined with a sensitivity of at least 0.81, or at least 0.82, or at least 0.83, or at least 0.84, or at least 0.85, or at least 0.86, or at least 0.87, or at least 0.88, or at least 0.89, or at least 0.90. In some embodiments, the rejection risk for the transplant recipient is determined with a specificity of at least 0.81, or at least 0.82, or at least 0.83, or at least 0.84, or at least 0.85, or at least 0.86, or at least 0.87, or at least 0.88, or at least 0.89, or at least 0.90. In some embodiments, the rejection risk for the transplant recipient is determined with an area under the curve (AUC) of at least at least 0.86, or at least 0.87, or at least 0.88, or at least 0.89, or at least 0.90, or at least 0.91 or at least 0.92, or at least 0.93, or at least 0.94, or at least 0.95.

### Methods for Measuring the Amount of Nucleic Acids

In some embodiments, the amount of cfDNA is measured by quantitative PCR. In some embodiments, the amount of cfDNA is measured by real-time PCR. In some embodiments, the amount of cfDNA is measured by digital PCR. In some embodiments, the amount of cfDNA is measured by sequencing such as high-throughput sequencing, next-generation sequence, or sequencing-by-synthesis.

In some embodiments, the amount of dd-cfDNA is determined by using ratiometric and/or machine learning-artificial intelligence comparisons at a single or a plurality of time points.

In some embodiments, the amount of cfDNA is measured by massively multiplex PCR (mmPCR) to obtain amplicons comprising biomarkers, and sequencing of the amplicons.

In some embodiments, the amount of cfDNA is measured by using microarray. In some embodiments, the amount of cfDNA is measured by using molecular barcodes and microscopic imaging (such as NanoString nCounter®).

In some embodiments, the amount of dd-cfDNA is measured by: extracting cfDNA from the biological sample of the pregnant transplant recipient, wherein the extracted cfDNA comprises dd-cfDNA, fd-cfDNA, and rd-cfDNA; performing targeted amplification of the extracted DNA at 10-50,000 target loci in a single reaction volume, selected based on homozygosity in the parents of the fetus; sequencing the amplified DNA by high-throughput sequencing to obtain sequencing reads and quantifying the amount of dd-cfDNA based on the sequencing reads.

In some embodiments, the method is performed without prior knowledge of donor genotypes. In some embodiments, the method does not comprise genotyping transplant donor(s). In some embodiments, the paternal genotype of the fetus is known. In some embodiments, the method comprises genotyping the biological father of the fetus.

In some embodiments, the amount of nucleic acids is measured by targeted amplification. In some embodiments, the amount of a particular cfDNA target is measured by targeted amplification. In some embodiments, the targeted amplification comprises PCR. In some embodiments, the primers for the targeted amplification include 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 pairs of forward and reverse PCR primers. In some embodiments, the targeted amplification comprises performing amplification at 100-20,000, 500-20,000, 1,000-10,000, 200-500, 500-1,000, 1,000-2,000, 2,000-5,000, 5,000-10,000, 10,000-20,000, 20,000-50,000 target loci in a single reaction volume using 500-20,000, 1,000-10,000, 200-500, 500-1,000, 1,000-2,000, 2,000-5,000, 5,000-10,000, 10,000-20,000, or 20,000-50,000 primer pairs to obtain amplification products.

In some embodiments, the targeted amplification comprises nested PCR. In some embodiments, the primers for the targeted amplification include a first universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target-specific primers, and a second universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 inner target-specific primers. In some embodiments, the targeted amplification comprises performing amplification at 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target loci in a single reaction volume using a first universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target-specific primers to obtain amplification products. In some embodiments, the targeted amplification comprises performing amplification at 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 target loci in a single reaction volume using a second universal primer and 10-50,000, 100-50,000, 200-50,000, 500-20,000, or 1,000-10,000, 200-500, 500-1,000, 1,000-2,000, 2,000-5,000, 5,000-10,000, 10,000-20,000, or 20,000-50,000 inner target-specific primers to obtain amplification products. In some embodiments, the methods disclosed herein comprise PCR amplification of at least 10, at least 100, at least 500, at least 1000, at least 2000 biomarkers, from 10-1000, 100-10000, 200-50000, or 500-20000 RNA biomarkers, using at least 10, at least 100, at least 500, at least 1000, at least 2000, from 10-1000, 100-10000, 200-50000, 500-20000 pairs of forward and reverse PCR primers. In some embodiments, step (b) comprises amplification of at least 2, at least 5, at least 10, at least 20, at least 30, at least 50, at least 100 target RNA molecules, from 2-10, 200-100, 50-500, or 50-2000 target RNA molecules, using at least at least 2, at least 5, at least 10, at least 20, at least 30, at least 50, at least 100 target RNA molecules, from 2-10, 200-100, 50-500 , or 50-2000 pairs of forward and reverse PCR primers.

In some embodiments, the method further comprises incorporating tags into the amplification products prior to performing high-throughput sequencing, wherein the tags comprise sequencing-compatible adaptors. In some embodiments, the method further comprises attaching tags to the extracted DNA prior to performing targeted amplification, wherein the tags comprise adaptors for amplification. In some embodiment, the tags comprise sample-specific barcodes, and wherein the method further comprises pooling the amplification products from a plurality of samples prior to high-throughput sequencing and sequencing the pool of amplification products together in a single run during the high-throughput sequencing.

In some embodiments, the amount of nucleic acids is determined by using for example, tracer nucleic acids, or internal calibration nucleic acids. The terms “tracer nucleic acids,” or “internal calibration nucleic acids” are used interchangeably and refer to a composition of nucleic acids for which one or more of the following is known advance—length, sequence, nucleotide composition, quantity, or biological origin. The tracer can be added to a biological sample derived from a subject to help estimate the amount of total cfDNA in said sample. It can also be added to reaction mixtures other than the biological sample itself.

### Cutoff Threshold for Determining Transplant Rejection

In some embodiments, the cutoff threshold is an estimate percentage of dd-cfDNA out of total cfDNA or a function thereof. In some embodiments, the cutoff threshold is 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, or 2.0% dd-cfDNA. In some embodiments, the cutoff threshold is adjusted according to the type of organs transplanted. In some embodiments, the cutoff threshold is adjusted according to the number of organs transplanted. In some embodiments, the cutoff threshold is adjusted according to the amount of fd-cfDNA in the sample.

In some embodiments, the cutoff threshold is proportional to an absolute dd-cfDNA concentration. In some embodiments, the cutoff threshold is a copy number of dd-cfDNA or a function thereof. In some embodiments, the cutoff threshold is expressed as quantity or absolute quantity of dd-cfDNA. In some embodiments, the cutoff threshold is expressed as quantity or absolute quantity of dd-cfDNA per volume unit of the blood sample. In some embodiments, the cutoff threshold is expressed as quantity or absolute quantity of dd-cfDNA per volume unit of the blood sample multiplied by body mass, BMI, or blood volume of the transplant recipient.

In some embodiments, the method further comprises measuring the amount of cfDNA and the amount of dd-cfDNA longitudinally for the same transplant recipient, and determining a longitudinal change in the amount of cfDNA or a function thereof and a longitudinal change in the amount of dd-cfDNA or a function thereof.

### Analysis of Donor-Derived Cell-Free DNA for Monitoring Transplant Rejection

“Acute rejection or AR” is the rejection by the immune system of a tissue transplant recipient when the transplanted tissue is immunologically foreign. Acute rejection is characterized by infiltration of the transplanted tissue by immune cells of the recipient, which carry out their effector function and destroy the transplanted tissue. The onset of acute rejection is rapid and generally occurs in humans within a few weeks after transplant surgery. Generally, acute rejection can be inhibited or suppressed with immunosuppressive drugs such as rapamycin, cyclosporin A, anti-CD40L monoclonal antibody and the like.

“Chronic transplant rejection or injury” or “CAI” generally occurs in humans within several months to years after engraftment, even in the presence of successful immunosuppression of acute rejection. Fibrosis is a common factor in chronic rejection of all types of organ transplants. Chronic rejection can typically be described by a range of specific disorders that are characteristic of the particular organ. For example, in lung transplants, such disorders include fibroproliferative destruction of the airway (bronchiolitis obliterans); in heart transplants or transplants of cardiac tissue, such as valve replacements, such disorders include fibrotic atherosclerosis; in kidney transplants, such disorders include, obstructive nephropathy, nephrosclerorsis, tubulointerstitial nephropathy; and in liver transplants, such disorders include disappearing bile duct syndrome. Chronic rejection can also be characterized by ischemic insult, denervation of the transplanted tissue, hyperlipidemia and hypertension associated with immunosuppressive drugs.

The term “transplant rejection” encompasses both acute and chronic transplant rejection. The term transplant rejection refers to a transplantation where the recipient is either the same species as the donor (“allograft rejection”), or a different species than the donor (“xenograft rejection”). The term “transplant injury” refers to all manners of graft dysfunction, irrespective of pathological diagnosis. The term “organ injury” refers to biomarkers that track with poor function of the organ, irrespective of the organ being native or a transplant, and irrespective of the etiology.

In one aspect, the present invention relates to a method of quantifying the amount of dd-cfDNA in a biological sample of a maternal transplant recipient, comprising: extracting DNA from the biological sample of the transplant recipient, wherein the DNA comprises dd-cfDNA, fd-cfDNA, and rd-cfDNA; preparing a composition from the cfDNA wherein one or more selected target loci are enriched, wherein the maternal transplant recipient and biological father of the fetus are homozygous at the selected target loci to ensure homozygosity of the fetus at the selected target loci, such that heterozygosity observed in the extracted cfDNA at the selected target loci originates from the transplant; and quantifying the amount of extracted cfDNA and the amount of dd-cfDNA based on heterozygosity at the selected target loci to determine whether the amount of dd-cfDNA or a function thereof exceeds a cutoff threshold indicating transplant rejection.

In one aspect, the present invention further comprises: measuring the amount of dd-cfDNA in a biological sample obtained from a maternal transplant recipient, extracting DNA from the sample obtained from the transplant recipient, wherein the extracted DNA comprises dd-cfDNA, fd-cfDNA, and rd-cfDNA; performing targeted amplification of the extracted DNA at 10-50,000 target loci in a single reaction volume; sequencing the amplified DNA to obtain sequencing reads and quantifying the amount of dd-cfDNA based on the sequencing reads, determining transplant rejection based on whether the amount of dd-cfDNA or a function thereof exceeds a cutoff threshold of cfDNA amount that indicates transplant rejection, wherein transplant rejection is determined based on whether the amount of dd-cfDNA or function thereof exceeds a cutoff threshold that indicates transplant rejection.

In another aspect, the present invention relates to a method of quantifying the amount of dd-cfDNA in a biological sample of a maternal transplant recipient, comprising: extracting DNA from the biological sample of the transplant recipient, wherein the DNA comprises dd-cfDNA, fd-cfDNA, and rd-cfDNA; performing targeted amplification at 10-50,000 or 500-50,000 target loci in a single reaction volume using 10-50,000 or 500-50,000 primer pairs, wherein the target loci comprise polymorphic loci and non-polymorphic loci, and wherein each primer pair is designed to amplify a target sequence of no more than 100 bp; and quantifying the amount of dd-cfDNA in the amplification products.

In another aspect, the present invention relates to a method of detecting dd-cfDNA in a biological sample of a maternal transplant recipient, comprising: extracting DNA from the biological sample of the transplant recipient, wherein the DNA comprises dd-cfDNA, fd-cfDNA, and rd-cfDNA; performing targeted amplification at 10-50,000 target loci in a single reaction volume using 10-50,000 primer pairs, wherein the target loci comprise polymorphic loci and non-polymorphic loci; sequencing the amplification products by high-throughput sequencing; and quantifying the amount of dd-cfDNA.

In a further aspect, the present invention relates to a method of determining the likelihood of transplant rejection within a maternal transplant recipient, the method comprising: extracting cfDNA from a biological sample of the transplant recipient, wherein the cfDNA comprises dd-cfDNA, fd-cfDNA, and rd-cfDNA; performing universal amplification of the extracted cfDNA; performing targeted amplification at 10-50,000 target loci in a single reaction volume using 10-50,000 primer pairs, wherein the target loci comprise polymorphic loci and non-polymorphic loci; sequencing the amplification products by high-throughput sequencing; and quantifying the amount of dd-cfDNA in the biological sample, wherein a greater amount of dd-cfDNA indicates a greater likelihood of transplant rejection.

In some embodiments, the method further comprises performing universal amplification of the extracted DNA. In some embodiments, the universal amplification preferentially amplifies dd-cf DNA over rd-cfDNA and fd-cfDNA that are disposed from bursting white-blood cells.

In some embodiments, the transplant recipient is a mammal. In some embodiments, the transplant recipient is a human. In some embodiments, the transplant donor is a human, a pig, a primate, a baboon, a cow, or a dog.

In some embodiments, the transplant recipient has received a transplant selected from organ transplant, tissue transplant, cell transplant, and fluid transplant. In some embodiments, the transplant recipient has received a transplant selected from kidney transplant, liver transplant, pancreas transplant, intestinal transplant, heart transplant, lung transplant, heart/lung transplant, stomach transplant, testis transplant, penis transplant, ovary transplant, uterus transplant, thymus transplant, face transplant, hand transplant, leg transplant, bone transplant, bone marrow transplant, cornea transplant, skin transplant, pancreas islet cell transplant, heart valve transplant, blood vessel transplant, and blood transfusion. In some embodiments, the transplant recipient has received SPK transplant.

In some embodiments, the quantifying step comprises determining the percentage of dd-cfDNA out of the total of dd-cfDNA, fd-cfDNA, and rd-cfDNA in the biological sample. In some embodiments, the quantifying step comprises determining the number of copies of dd-cfDNA per volume unit of the blood sample.

In some embodiments, the method further comprises detecting the occurrence or likely occurrence of active rejection of transplantation using the quantified amount of dd-cfDNA. In some embodiments, the method is performed without prior knowledge of donor genotypes.

In some embodiments, the targeted amplification comprises simultaneously amplifying 10-50,000 target loci in a single reaction volume using (i) at least 10-50,000 different primer pairs, or (ii) at least 10-50,000 target-specific primers and a universal or tag-specific primer 10-50,000 primer pairs.

In some embodiments, each primer pair is designed to amplify a target sequence of about 50-100 bp. In some embodiments, each primer pair is designed to amplify a target sequence of no more than 75 bp. In some embodiments, each primer pair is designed to amplify a target sequence of about 60-75 bp. In some embodiments, each primer pair is designed to amplify a target sequence of about 65 bp.

In some embodiments, the targeted amplification comprises amplifying at least 10 target loci in a single reaction volume. In some embodiments, the targeted amplification comprises amplifying at least 100 target loci in a single reaction volume. In some embodiments, the targeted amplification comprises amplifying at least 1,000 target loci in a single reaction volume. In some embodiments, the targeted amplification comprises amplifying at least 2,000 target loci in a single reaction volume. In some embodiments, the targeted amplification comprises amplifying at least 5,000 target loci in a single reaction volume. In some embodiments, the targeted amplification comprises amplifying at least 10,000 target loci in a single reaction volume. In some embodiments, the targeted amplification comprises amplifying 10 to 10,000, 10 to 50,000, 100 to 50,000, or 1000 to 50,000 target loci in a single reaction volume.

In some embodiments, method further comprises measuring an amount of one or more alleles at the target loci that are polymorphic loci. In some embodiments, the polymorphic loci and the non-polymorphic loci are amplified in a single reaction.

In some embodiments, the quantifying step comprises detecting the amplified target loci using a microarray. In some embodiments, the quantifying step does not comprise using a microarray.

In some embodiments, the targeted amplification comprises simultaneously amplifying 10-50,000 target loci in a single reaction volume using (i) at least 10-50,000 different primer pairs, or (ii) at least 10-50,000 target-specific primers and a universal or tag-specific primer 10-50,000 primer pairs.

In a further aspect, the present invention relates to a method of diagnosing a transplant within a maternal transplant recipient as undergoing acute rejection, the method comprising: extracting DNA from the biological sample of the maternal transplant recipient, wherein the DNA comprises dd-cfDNA and rd-cfDNA; performing universal amplification of the extracted DNA; performing targeted amplification at 100-50,000 target loci in a single reaction volume using 100-50,000 primer pairs, wherein the target loci comprise polymorphic loci and non-polymorphic loci; sequencing the amplification products by high-throughput sequencing; and quantifying the amount of dd-cfDNA in the biological sample, wherein an amount of dd-cfDNA of greater than 1% (or 0.5%, or 0.6%, or 0.7%, or 0.8%, or 0.9%, or 1.1%, or 1.2%, or 1.3%, or 1.4%, or 1.5%, or 1.6%, or 1.7%, or 1.8%, or 1.9%, or 2.0%) indicates that the transplant is undergoing acute rejection.

In some embodiments, the transplant rejection is antibody mediated transplant rejection. In some embodiments, the transplant rejection is T cell mediated transplant rejection.

In some embodiments, an amount of dd-cfDNA of less than 1% (or 0.9 %, or 0.8%, or 0.7%, or 0.6%, or 0.5%) indicates that the transplant is either undergoing borderline rejection, undergoing other injury, or stable.

In a further aspect, the present invention relates to a method of monitoring immunosuppressive therapy in a subject, the method comprising: extracting DNA from the biological sample of the transplant recipient, wherein the DNA comprises dd-cfDNA, rd-cfDNA, and fd-cfDNA; performing universal amplification of the extracted DNA; performing targeted amplification at 10-50,000 target loci in a single reaction volume using 10-50,000 primer pairs, wherein the target loci comprise polymorphic loci and non-polymorphic loci; sequencing the amplification products by high-throughput sequencing; and quantifying the amount of dd-cfDNA in the biological sample, wherein a change in levels of dd-cfDNA over a time interval is indicative of transplant status.

In some embodiments, the method further comprising adjusting immunosuppressive therapy based on the levels of dd-cfDNA over the time interval.

In some embodiments, an increase in the levels of dd-cfDNA is indicative of transplant rejection and a need for adjusting immunosuppressive therapy. In some embodiments, no change or a decrease in the levels of dd-cfDNA indicates transplant tolerance or stability, and a need for adjusting immunosuppressive therapy.

In some embodiments, the method does not comprise genotyping the transplant donor and/or the transplant recipient.

In some embodiments, the target loci comprise at least 10 polymorphic loci, or at least 100 polymorphic loci, or at least 1,000 polymorphic loci, or at least 2,000 polymorphic loci, or at least 5,000 polymorphic loci, or at least 10,000 polymorphic loci.

In some embodiments, the extracting step comprises size selection to enrich for dd-cfDNA and reduce the amount of rd-cfDNA disposed from bursting white-blood cells.

In some embodiments, the universal amplification step preferentially amplifies dd-cfDNA over rd-cfDNA or fd-cfDNA.

In some embodiments, the method comprises longitudinally collecting a plurality of blood samples from the transplant recipient after transplantation, and measuring the amount of cfDNA and dd-cfDNA to determine a longitudinal change in the amount of cfDNA or a function thereof and a longitudinal change in the amount of dd-cfDNA for the transplant recipient. In some embodiments, the method comprises collecting and analyzing blood samples from the transplant recipient for a time period of about three months, or about six months, or about twelve months, or about eighteen months, or about twenty-four months, etc. In some embodiments, the method comprises collecting blood samples from the transplant recipient at an interval of about one week, or about two weeks, or about three weeks, or about one month, or about two months, or about three months, etc.

In some embodiments, the method further comprises titrating the dosage of the immunosuppressive therapy according to the longitudinal change in the total amount of cfDNA or a function thereof and the longitudinal change in the amount of dd-cfDNA or a function thereof.

In one aspect the present disclosure relates to a method of administrating immunosuppressive therapy in a maternal transplant recipient, comprising: measuring the amount of cfDNA in a biological sample of the transplant recipient; measuring the amount of dd-cfDNA in a biological sample of the transplant recipient; and titrating the dosage of an immunosuppressive therapy according to the amount of cfDNA or a function thereof and the amount of dd-cfDNA or a function thereof.

In some embodiments, the method has a sensitivity of at least 80%, or at least 85%, or at least 90%, or at least 95%, or at least 98% in identifying acute rejection (AR) over non-AR with a cutoff threshold of 1% dd-cfDNA and a confidence interval of 95%.

In some embodiments, the method has a specificity of at least 60%, or at least 65%, or at least 70%, or at least 75%, or at least 80%, or at least 85%, or at least 90% in identifying AR over non-AR with a cutoff threshold of 1% dd-cfDNA and a confidence interval of 95%.

In some embodiments, the method has an area under the curve (AUC) of at least 0.8, or 0.85, or at least 0.9, or at least 0.95 in identifying AR over non-AR with a cutoff threshold of 1% dd-cfDNA and a confidence interval of 95%.

In some embodiments, the method has a sensitivity of at least 80%, or at least 85%, or at least 90%, or at least 95%, or at least 98% in identifying AR over normal, stable allografts (STA) with a cutoff threshold of 1% dd-cfDNA and a confidence interval of 95%.

In some embodiments, the method has a specificity of at least 80%, or at least 85%, or at least 90%, or at least 95%, or at least 98% in identifying AR over STA with a cutoff threshold of 1% dd-cfDNA and a confidence interval of 95%.

In some embodiments, the method has an AUC of at least 0.8, or 0.85, or at least 0.9, or at least 0.95, or at least 0.98, or at least 0.99 in identifying AR over STA with a cutoff threshold of 1% dd-cfDNA and a confidence interval of 95%.

In some embodiments, the method has a sensitivity as determined by a limit of blank (LoB) of 0.5% or less, and a limit of detection (LoD) of 0.5% or less. In some embodiments, LoB is 0.23% or less and LoD is 0.29% or less. In some embodiments, the sensitivity is further determined by a limit of quantitation (LoQ). In some embodiments, LoQ is 10 times greater than the LoD; LoQ may be 5 times greater than the LoD; LoQ may be 1.5 times greater than the LoD; LoQ may be 1.2 times greater than the LoD; LoQ may be 1.1 times greater than the LoD; or LoQ may be equal to or greater than the LoD. In some embodiments, LoB is equal to or less than 0.04%, LoD is equal to or less than 0.05%, and/or LoQ is equal to the LoD.

In some embodiments, the method has an accuracy as determined by evaluating a linearity value obtained from linear regression analysis of measured donor fractions as a function of the corresponding attempted spike levels, wherein the linearity value is a R² value, wherein the R² value is from about 0.98 to about 1.0. In some embodiments, the R² value is 0.999. In some embodiments, the method has an accuracy as determined by using linear regression on measured donor fractions as a function of the corresponding attempted spike levels to calculate a slope value and an intercept value, wherein the slope value is from about 0.9 to about 1.2 and the intercept value is from about −0.0001 to about 0.01. In some embodiments, the slope value is approximately 1, and the intercept value is approximately 0.

In some embodiments, the method has a precision as determined by calculating a coefficient of variation (CV), wherein the CV is less than about 10.0%. CV is less than about 6%. In some embodiments, the CV is less than about 4%. In some embodiments, the CV is less than about 2%. In some embodiments, the CV is less than about 1%.

In some embodiments, the AR is antibody-mediated rejection (ABMR). In some embodiments, the AR is T-cell-mediated rejection (TCMR).

In some embodiments, disclosed herein is a method of amplifying target loci of dd-cfDNA from a biological sample of a maternal transplant recipient, the method comprising: extracting DNA from the blood sample of the transplant recipient, wherein the DNA comprises DNA derived from the transplanted cells, from the transplant recipient, and from the fetus; enriching the extracted DNA at target loci, wherein the target loci comprise 10 to 50,000 target loci comprising polymorphic loci and non-polymorphic loci; and amplifying the target loci.

In some embodiments, disclosed herein is a method of detecting dd-cfDNA in a blood sample from a maternal transplant recipient, the method comprising: a) extracting DNA from the blood sample of the transplant recipient, wherein the DNA comprises cfDNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; d) contacting the amplified target loci with probes that specifically hybridize to target loci; and e) detecting binding of the target loci with the probes, thereby detecting dd-cfDNA in the blood sample. In some embodiments, the probes arc labelled with a detectable marker.

In some embodiments, disclosed herein is a method of determining the likelihood of transplant rejection within a maternal transplant recipient, the method comprising: a) extracting DNA from the blood sample of the transplant recipient, wherein the DNA comprises cfDNA derived from both the transplanted cells and from the transplant recipient, b) enriching the extracted DNA at target loci, wherein the target loci comprise 50 to 5000 target loci comprising polymorphic loci and non-polymorphic loci; c) amplifying the target loci; and d) measuring an amount of transplant DNA and an amount of transplant recipient DNA in the recipient blood sample; wherein a greater amount of dd-cfDNA indicates a greater likelihood of transplant rejection.

In some embodiments, disclosed herein is a method of monitoring immunosuppressive therapy in a subject, the method comprising a) extracting DNA from the biological sample of the transplant recipient, wherein the DNA comprises cfDNA derived from both the transplanted cells and from the transplant recipient and from the fetus, b) enriching the extracted DNA at target loci, wherein the target loci comprise 10 to 50,000 target loci comprising polymorphic loci and non-polymorphic loci; c) amplifying the target loci; and d) measuring an amount of transplant DNA and an amount of recipient and fetal DNA in the recipient biological sample; wherein a change in levels of dd-cfDNA over a time interval is indicative of transplant status. In some embodiments, the method further comprises adjusting immunosuppressive therapy based on the levels of dd-cfDNA over the time interval. In some embodiments, an increase in the levels of dd-cfDNA are indicative of transplant rejection and a need for adjusting immunosuppressive therapy. In some embodiments, a change or a decrease in the levels of dd-cfDNA indicates transplant tolerance or stability, and a need for adjusting immunosuppressive therapy.

In some embodiments, the methods disclosed herein require that the target loci are homozygous in the maternal transplant recipient and the biological father of the fetus, ensuring homozygosity in the fetus at the target loci. Any heterozygosity detected in the biological sample from the maternal transplant recipient will originate from the donor. Heterozygosity at the target loci can therefore be used to quantify the amount of dd-cfDNA present in the biological sample.

### Analytical Methods

In some embodiments, the method also includes obtaining genotypic data from one or more of the transplant donor, the maternal transplant recipient, the fetus, and the biological father of the fetus. In some embodiments, obtaining genotypic data from one or more of the transplant donor, the maternal transplant recipient, the fetus, and the biological father of the fetus includes preparing the DNA from the donor, recipient, fetus, and father, where the preparing comprises preferentially enriching the DNA at the plurality of polymorphic loci to give prepared DNA, optionally amplifying the prepared DNA, and measuring the DNA in the prepared sample at the plurality of polymorphic loci. The genotypic data can be used to determine loci at which the maternal transplant recipient and the biological father of the fetus are homozygous.

In some embodiments, building a joint distribution model for the expected allele count probabilities of the plurality of polymorphic loci on the chromosome is done using the obtained genetic data from the one or more of the transplant donor, the maternal transplant recipient, the fetus, and the biological father of the fetus. In some embodiments, the first sample has been isolated from transplant recipient plasma and where the obtaining genotypic data from the transplant recipient is done by estimating the recipient genotypic data from the DNA measurements made on the prepared sample.

In some embodiments, preferential enrichment results in average degree of allelic bias between the prepared sample and the first sample of a factor selected from the group consisting of no more than a factor of 2, no more than a factor of 1.5, no more than a factor of 1.2, no more than a factor of 1.1, no more than a factor of 1.05, no more than a factor of 1.02, no more than a factor of 1.01, no more than a factor of 1.005, no more than a factor of 1.002, no more than a factor of 1.001 and no more than a factor of 1.0001. In some embodiments, the plurality of polymorphic loci are SNPs. In some embodiments, measuring the DNA in the prepared sample is done by sequencing.

In some embodiments, a diagnostic box is disclosed for helping to determine transplant status in a maternal transplant recipient where the diagnostic box is capable of executing the preparing and measuring steps of the disclosed methods.

In some embodiments, the allele counts are probabilistic rather than binary. In some embodiments, measurements of the DNA in the prepared sample at the plurality of polymorphic loci are also used to determine whether or not the transplant has inherited one or a plurality of linked haplotypes.

In some embodiments, building a joint distribution model for allele count probabilities is done by using data about the probability of chromosomes crossing over at different locations in a chromosome to model dependence between polymorphic alleles on the chromosome. In some embodiments, building a joint distribution model for allele counts and the step of determining the relative probability of each hypothesis are done using a method that does not require the use of a reference chromosome.

In some embodiments, determining the relative probability of each hypothesis makes use of an estimated fraction of dd-cfDNA in the prepared sample. In some embodiments, the DNA measurements from the prepared sample used in calculating allele count probabilities and determining the relative probability of each hypothesis comprise primary genetic data. In some embodiments, selecting the transplant status corresponding to the hypothesis with the greatest probability is carried out using maximum likelihood estimates or maximum a posteriori estimates.

In some embodiments, calling the transplant status also includes combining the relative probabilities of each of the status hypotheses determined using the joint distribution model and the allele count probabilities with relative probabilities of each of the status hypotheses that are calculated using statistical techniques taken from a group consisting of a read count analysis, comparing heterozygosity rates, a statistic that is only available when donor genetic information is used, the probability of normalized genotype signals for certain donor/recipient contexts, a statistic that is calculated using an estimated transplant fraction of the first sample or the prepared sample, and combinations thereof.

In some embodiments, a confidence estimate is calculated for the called transplant status. In some embodiments, the method also includes taking a clinical action based on the called transplant status.

In some embodiments, a report displaying a determined transplant status is generated using the method. In some embodiments, a kit is disclosed for determining a transplant status designed to be used with the methods disclosed herein, the kit including a plurality of inner forward primers and optionally the plurality of inner reverse primers, where each of the primers is designed to hybridize to the region of DNA immediately upstream and/or downstream from one of the polymorphic sites on the target chromosome, and optionally additional chromosomes, where the region of hybridization is separated from the polymorphic site by a small number of bases, where the small number is selected from the group consisting of 1, 2, 3, 4, 5, 6 to 10, 11 to 15, 16 to 20, 21 to 25, 26 to 30, 31 to 60, and combinations thereof.

In some embodiments, the cutoff threshold value takes into account one or more of the followings: donor genome copies per volume of plasma, cfDNA yield per volume of plasma, fetal genome copies per volume of plasma, number of fetus' present, stage of pregnancy, donor height, donor weight, donor age, donor gender, donor ethnicity, donor organ mass, donor organ, live vs deceased donor, related vs unrelated donor, recipient height, recipient weight, recipient age, recipient gender, recipient ethnicity, creatinine, eGFR (estimated glomerular filtration rate), cfDNA methylation, DSA (donor-specific antibodies), KDPI (kidney donor profile index), medications (immunosuppression, steroids, blood thinners, etc.), infections (BKV, EBV, CMV, UTI), recipient and/or donor HLA alleles or epitope mismatches, Banff classification of renal transplant pathology, and for-cause vs surveillance or protocol biopsy.

In some embodiments, the cutoff threshold value is scaled according to the amount of total cfDNA in the blood sample.

In some embodiments, the method has a sensitivity of at least 80% in identifying acute rejection (AR) over non-AR when the dd-cfDNA amount is above the cutoff threshold value scaled according to the amount of total cfDNA in the blood sample and a confidence interval of 95%.

In some embodiments, the method has a specificity of at least 70% in identifying acute rejection (AR) over non-AR when the dd-cfDNA amount is above the cutoff threshold value scaled according to the amount of total cfDNA in the blood sample and a confidence interval of 95%.

In some embodiments, the method has a sensitivity of at least 80% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 85% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 90% in identifying acute rejection (AR) over non-AR when the dd-cfDNA amount is above the cutoff threshold value scaled according to the amount of total cfDNA in the blood sample and a confidence interval of 95%. In some embodiments, the method has a sensitivity of at least 95% in identifying acute rejection (AR) over non-AR when the dd-cfDNA amount is be above the cutoff threshold value scaled according to the amount of total cfDNA in the blood sample and a confidence interval of 95%.

In some embodiments, the method has a specificity of at least 70% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 75% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 85% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 90% in identifying acute rejection (AR) over non-AR when the dd-cfDNA amount is above the cutoff threshold value scaled according to the amount of total cfDNA in the blood sample and a confidence interval of 95%. In some embodiments, the method has a specificity of at least 95% in identifying acute rejection (AR) over non-AR when the dd-cfDNA amount is above the cutoff threshold value scaled according to the amount of total cfDNA in the blood sample and a confidence interval of 95%.

### Multiplex Amplification

In some embodiments, the method comprises performing a multiplex amplification reaction to amplify a plurality of target loci in one reaction mixture before determining the sequences of the selectively enriched DNA. In some embodiments, the target loci are selected at loci at which both the maternal transplant recipient and biological father of the fetus are homozygous.

In certain illustrative embodiments, the nucleic acid sequence data is generated by performing high throughput sequencing of a plurality of copies of a series of amplicons generated using a multiplex amplification reaction, wherein each amplicon of the series of amplicons spans at least one polymorphic locus of the set of polymorphic loci and wherein each of the polymeric loci of the set is amplified. For example, in these embodiments a multiplex PCR to amplify amplicons across at least 10; 100; 200; 500; 1,000; 2,000; 5,000; 10,000; 20,000; 50,000; or 100,000 polymorphic loci (e.g., SNP loci) may be performed. This multiplex reaction can be set up as a single reaction or as pools of different subset multiplex reactions. The multiplex reaction methods provided herein, such as the massive multiplex PCR disclosed herein provide an exemplary process for carrying out the amplification reaction to help attain improved multiplexing and therefore, sensitivity levels.

In some embodiments, amplification is performed using direct multiplexed PCR, sequential PCR, nested PCR, doubly nested PCR, one-and-a-half sided nested PCR, fully nested PCR, one sided fully nested PCR, one-sided nested PCR, hemi-nested PCR, hemi-nested PCR, triply hemi-nested PCR, semi-nested PCR, one sided semi-nested PCR, reverse semi-nested PCR method, or one-sided PCR, which are described in U.S. application Ser. No. 13/683,604, filed Nov. 21, 2012, U.S. Publication No. 2013/0123120, U.S. application Ser. No. 13/300,235, filed Nov. 18, 2011, U.S. Publication No 2012/0270212, and U.S. Ser. No. 61/994,791, filed May 16, 2014, all of which are hereby incorporated by reference in their entirety.

In some embodiments, multiplex PCR is used. In some embodiments, the method of amplifying target loci in a nucleic acid sample involves (i) contacting the nucleic acid sample with a library of primers that simultaneously hybridize to at least 10; 100; 200; 500; 1,000; 2,000; 5,000; 10,000; 20,000; 50,000; or 100,000 different target loci to produce a single reaction mixture; and (ii) subjecting the reaction mixture to primer extension reaction conditions (such as PCR conditions) to produce amplified products that include target amplicons. In some embodiments, at least 50, 60, 70, 80, 90, 95, 96, 97, 98, 99, or 99.5% of the targeted loci are amplified. In various embodiments, less than 60, 50, 40, 30, 20, 10, 5, 4, 3, 2, 1, 0.5, 0.25, 0.1, or 0.05% of the amplified products are primer dimers. In some embodiments, the primers are in solution (such as being dissolved in the liquid phase rather than in a solid phase). In some embodiments, the primers are in solution and are not immobilized on a solid support. In some embodiments, the primers are not part of a microarray.

In certain embodiments, the multiplex amplification reaction is performed under limiting primer conditions for at least ½ of the reactions. In some embodiments, limiting primer concentrations are used in 1/10, ⅕, ¼, ⅓, ½, or all of the reactions of the multiplex reaction. Provided herein are factors to consider in achieving limiting primer conditions in an amplification reaction such as PCR.

In certain embodiments, the multiplex amplification reaction can include, for example, between 2,500 and 50,000 multiplex reactions. In certain embodiments, the following ranges of multiplex reactions are performed: between 10, 100, 200, 250, 500, 1,000, 2,500, 5,000, 10,000, 20,000, 25000, 50,000 on the low end of the range and between 200, 250, 500, 1,000, 2,500, 5,000, 10,000, 20,000, 25,000, 50,000, and 100,000 on the high end of the range.

In an embodiment, a multiplex PCR assay is designed to amplify potentially heterozygous SNP or other polymorphic or non-polymorphic loci on one or more chromosomes and these assays are used in a single reaction to amplify DNA. The number of PCR assays may be between 10 and 200 PCR assays, between 200 and 1,000 PCR assays, between 1,000 and 5,000 PCR assays, or between 5,000 and 20,000 PCR assays (10 to 200-plex, 200 to 1,000-plex, 1,000 to 5,000-plex, 5,000 to 20,000-plex, more than 20,000-plex respectively). In an embodiment, a multiplex pool of at least 10,000 PCR assays (10,000-plex) are designed to amplify potentially heterozygous SNP loci a single reaction to amplify cfDNA obtained from a blood, plasma, serum, solid tissue, or urine sample. The SNP frequencies of each locus may be determined by clonal or some other method of sequencing of the amplicons. In another embodiment the original cfDNA samples is split into two samples and parallel 5,000-plex assays are performed. In another embodiment the original cfDNA samples is split into n samples and parallel (~10,000/n)-plex assays are performed where n is between 2 and 12, or between 12 and 24, or between 24 and 48, or between 48 and 96.

In an embodiment, a method disclosed herein uses highly efficient highly multiplexed targeted PCR to amplify DNA followed by high throughput sequencing to determine the allele frequencies at each target locus. One technique that allows highly multiplexed targeted PCR to perform in a highly efficient manner involves designing primers that are unlikely to hybridize with one another. The PCR probes, typically referred to as primers, are selected by creating a thermodynamic model of potentially adverse interactions between at least 10, at least 100, at least 200, at least 500, at least 1,000, at least 2,000, at least 5,000, at least 10,000, at least 20,000, or at least 50,000 potential primer pairs, or unintended interactions between primers and sample DNA, and then using the model to eliminate designs that are incompatible with other the designs in the pool. Another technique that allows highly multiplexed targeted PCR to perform in a highly efficient manner is using a partial or full nesting approach to the targeted PCR. Using one or a combination of these approaches allows multiplexing of at least 10, at least 100, at least 200, at least 500, at least 1,000, at least 2,000, at least 5,000, at least 10,000, at least 20,000, or at least 50,000 primers in a single pool with the resulting amplified DNA comprising a majority of DNA molecules that, when sequenced, will map to targeted loci. Using one or a combination of these approaches allows multiplexing of a large number of primers in a single pool with the resulting amplified DNA comprising greater than 50%, greater than 80%, greater than 90%, greater than 95%, greater than 98%, or greater than 99% DNA molecules that map to targeted loci.

Bioinformatics methods are used to analyze the genetic data obtained from multiplex PCR. The bioinformatics methods useful and relevant to the methods disclosed herein can be found in U.S. Patent Publication No. 2018/0025109, incorporated by reference herein.

### High-Throughput Sequencing

In some embodiments, the sequences of the amplicons are determined by performing high-throughput sequencing.

The genetic data of the transplant recipient and/or of the transplant donor can be transformed from a molecular state to an electronic state by measuring the appropriate genetic material using tools and or techniques taken from a group including, but not limited to: genotyping microarrays, and high throughput sequencing. Some high throughput sequencing methods include Sanger DNA sequencing, pyrosequencing, the ILLUMINA SOLEXA platform, ILLUMINA's GENOME ANALYZER, or APPLIED BIOSYSTEM's 454 sequencing platform, HELICOS's TRUE SINGLE MOLECULE SEQUENCING platform, HALCYON MOLECULAR's electron microscope sequencing method, PacBio, Oxford Nanopore, or any other sequencing method. In some embodiments, the high throughput sequencing is performed on Illumina NextSeq®. All of these methods physically transform the genetic data stored in a sample of DNA into a set of genetic data that is typically stored in a memory device en route to being processed.

In some embodiments, the sequences of the selectively enriched DNA are determined by performing microarray analysis. In an embodiment, the microarray may be an ILLUMINA SNP microarray, or an AFFYMETRIX SNP microarray.

In some embodiments, the sequences of the selectively enriched DNA are determined by performing quantitative PCR (qPCR) or digital droplet PCR (ddPCR) analysis. qPCR measures the intensity of fluorescence at specific times (generally after every amplification cycle) to determine the relative amount of target molecule (DNA). ddPCR measures the actual number of molecules (target DNA) as each molecule is in one droplet, thus making it a discrete “digital” measurement. It provides absolute quantification because ddPCR measures the positive fraction of samples, which is the number of droplets that are fluorescing due to proper amplification. This positive fraction accurately indicates the initial amount of template nucleic acid.

### Definitions

As used herein the term “single nucleotide polymorphism (SNP)” refers to a single nucleotide that may differ between the genomes of two members of the same species. The usage of the term does not imply any limit on the frequency with which each variant occurs.

In some embodiments, for example, sequence refers to a DNA or RNA sequence or a genetic sequence. It may refer to the primary, physical structure of the DNA or RNA molecule or strand in an individual. It may refer to the sequence of nucleotides found in that DNA or RNA molecule, or the complementary strand to the DNA or RNA molecule. It may refer to the information contained in the DNA or RNA molecule as its representation in silico.

In some embodiments, for example, locus refers to a particular region of interest on the DNA or RNA of an individual and includes without limitation one or more SNPs, the site of a possible insertion or deletion, or the site of some other relevant genetic variation. Disease-linked SNPs may also refer to disease-linked loci.

In some embodiments, for example, polymorphic allele, also “polymorphic locus,” refers to an allele or locus where the genotype varies between individuals within a given species. Some examples of polymorphic alleles include single nucleotide polymorphisms (SNPs), short tandem repeats, deletions, duplications, and inversions.

In some embodiments, for example, allele refers to the nucleotides or nucleotide sequence occupying a particular locus.

In some embodiments, for example, genetic data also “genotypic data” refers to the data describing aspects of the genome of one or more individuals. It may refer to one or a set of loci, partial or entire sequences, partial or entire chromosomes, or the entire genome. It may refer to the identity of one or a plurality of nucleotides; it may refer to a set of sequential nucleotides, or nucleotides from different locations in the genome, or a combination thereof. Genotypic data is typically in silico, however, it is also possible to consider physical nucleotides in a sequence as chemically encoded genetic data. Genotypic Data may be said to be “on,” “of,” “at,” “from” or “on” the individual(s). Genotypic Data may refer to output measurements from a genotyping platform where those measurements are made on genetic material.

In some embodiments, for example, genetic material also “genetic sample” refers to physical matter, such as tissue or blood, from one or more individuals comprising nucleic acids (e.g., comprising DNA or RNA)

In some embodiments, for example, allelic data refers to a set of genotypic data concerning a set of one or more alleles. It may refer to the phased, haplotypic data. It may refer to SNP identities, and it may refer to the sequence data of the nucleic acid, including insertions, deletions, repeats and mutations.

In some embodiments, for example, allelic state refers to the actual state of the genes in a set of one or more alleles. It may refer to the actual state of the genes described by the allelic data.

In some embodiments, for example, allelic ratio or allele ratio, refers to the ratio between the amount of each allele at a locus that is present in a sample or in an individual. When the sample was measured by sequencing, the allelic ratio may refer to the ratio of sequence reads that map to each allele at the locus. When the sample was measured by an intensity based measurement method, the allele ratio may refer to the ratio of the amounts of each allele present at that locus as estimated by the measurement method.

In some embodiments, for example, allele count refers to the number of sequences that map to a particular locus, and if that locus is polymorphic, it refers to the number of sequences that map to each of the alleles. If each allele is counted in a binary fashion, then the allele count will be whole number. If the alleles are counted probabilistically, then the allele count can be a fractional number.

In some embodiments, for example, primer, also “PCR probe” refers to a single DNA molecule (a DNA oligomer) or a collection of DNA molecules (DNA oligomers) where the DNA molecules are identical, or nearly so, and where the primer contains a region that is designed to hybridize to a targeted polymorphic locus, and contain a priming sequence designed to allow amplification such as PCR amplification. A primer may also contain a molecular barcode. A primer may contain a random region that differs for each individual molecule.

In some embodiments, for example, hybrid capture probe refers to any nucleic acid sequence, possibly modified, that is generated by various methods such as PCR or direct synthesis and intended to be complementary to one strand of a specific target DNA or RNA sequence in a sample. The exogenous hybrid capture probes may be added to a prepared sample and hybridized through a denaturation-reannealing process to form duplexes of exogenous-endogenous fragments. These duplexes may then be physically separated from the sample by various means.

In some embodiments, for example, sequence read refers to data representing a sequence of nucleotide bases that were measured using a clonal sequencing method. Clonal sequencing may produce sequence data representing single, or clones, or clusters of one original DNA or RNA molecule. A sequence read may also have associated quality score at each base position of the sequence indicating the probability that nucleotide has been called correctly.

In some embodiments, for example, mapping a sequence read is the process of determining a sequence read's location of origin in the genome sequence of a particular organism. The location of origin of sequence reads is based on similarity of nucleotide sequence of the read and the genome sequence.

In some embodiments, for example, DNA or RNA of donor origin refers to DNA or RNA that was originally part of a cell whose genotype was essentially equivalent to that of the transplant donor. The donor can be a human or a non-human mammal (e.g., pig).

In some embodiments, for example, DNA or RNA of recipient origin refers to DNA or RNA that was originally part of a cell whose genotype was essentially equivalent to that of the transplant recipient.

In some embodiments, DNA may refer to genomic DNA, cDNA, cell-free DNA (cfDNA), cell-free mitochondrial DNA (cf mDNA), cell-free DNA that originated from nuclear DNA (cf nDNA), cellular DNA, or mitochondrial DNA. In some embodiments, the cfDNA is derived from exosomes or microvesicles.

In some embodiments, RNA may refer to messenger RNA (mRNA), small non-coding RNA (sncRNA), transfer RNA (tRNA), or a non-protein coding RNA from cells. In some embodiments, sncRNA comprises micro RNA (miRNA), piwi-interacting RNA (piRNA), small nucleolar RNA (snoRNA), small nuclear RNA (snRNA), or miscellaneous RNA (miscRNA). In some embodiments, the RNA is cell-free RNA. In some embodiments, the cell-free RNA is derived from exosomes or microvesicles.

In some embodiments, amplification of RNA comprises reverse transcription of RNA to produce complementary DNA (cDNA) followed by amplification of cDNA by amplification methods disclosed elsewhere herein.

In some embodiments, for example, transplant recipient plasma refers to the plasma portion of the blood from a female patient who has received an allograft or xenograft, e.g., an organ transplant recipient.

As used herein, “maternal transplant recipient”, regnant transplant recipient”, and “transplant recipient” are used interchangeably. In some embodiments, maternal transplant recipient refers to a pregnant patient who has received an allograft or xenograft, e.g., an organ transplant recipient. In some embodiments, the maternal transplant recipient may no longer be pregnant but fetal derived nucleic acids, such as fd-cfDNA, may still be present in the maternal transplant recipient. In some embodiments, the biological father of the fetus is the father of the fetus of the maternal transplant recipient. In some embodiments, paternal DNA refers to DNA from the biological father of the fetus.

In some embodiments, a biological sample may be blood, plasma, saliva, semen, sperm, cell culture supernatant, mucus secretion, dental plaque, gastrointestinal tract tissue, stool, urine, hair, bone, body fluids, tears, tissue, skin, fingernails, blastomeres, embryos, amniotic fluid, chorionic villus samples, bile, lymph, cervical mucus, or a forensic sample.

In some embodiments, for example, preferential enrichment of DNA or RNA that corresponds to a locus, or preferential enrichment of DNA or RNA at a locus, refers to any technique that results in the percentage of molecules of DNA or RNA in a post-enrichment DNA or RNA mixture that correspond to the locus being higher than the percentage of molecules of DNA or RNA in the pre-enrichment DNA or RNA mixture that correspond to the locus. The technique may involve selective amplification of DNA or RNA molecules that correspond to a locus. The technique may involve removing DNA or RNA molecules that do not correspond to the locus. The technique may involve a combination of methods. The degree of enrichment is defined as the percentage of molecules of DNA or RNA in the post-enrichment mixture that correspond to the locus divided by the percentage of molecules of DNA or RNA in the pre-enrichment mixture that correspond to the locus. Preferential enrichment may be carried out at a plurality of loci. In some embodiments of the present disclosure, the degree of enrichment is greater than 20. In some embodiments of the present disclosure, the degree of enrichment is greater than 200. In some embodiments of the present disclosure, the degree of enrichment is greater than 2,000. When preferential enrichment is carried out at a plurality of loci, the degree of enrichment may refer to the average degree of enrichment of all of the loci in the set of loci.

In some embodiments, for example, amplification refers to a technique that increases the number of copies of a molecule of DNA and/or RNA.

In some embodiments, for example, selective amplification may refer to a technique that increases the number of copies of a particular molecule of DNA and/or RNA, or molecules of DNA and/or RNA that correspond to a particular region of DNA and/or RNA. It may also refer to a technique that increases the number of copies of a particular targeted molecule of DNA and/or RNA, or targeted region of DNA and/or RNA more than it increases non-targeted molecules or regions of DNA and/or RNA. Selective amplification may be a method of preferential enrichment.

In some embodiments, for example, universal priming sequence refers to a DNA sequence that may be appended to a population of target nucleic acid molecules, for example by ligation, PCR, or ligation mediated PCR. Once added to the population of target molecules, primers specific to the universal priming sequences can be used to amplify the target population using a single pair of amplification primers. Universal priming sequences need not be related to the target sequences.

In some embodiments, for example, universal adapters, or ‘ligation adaptors’ or ‘library tags’ arc DNA molecules containing a universal priming sequence that can be covalently linked to the 5-prime and 3-prime end of a population of target double stranded DNA molecules. The addition of the adapters provides universal priming sequences to the 5-prime and 3-prime end of the target population from which PCR amplification can take place, amplifying all molecules from the target population, using a single pair of amplification primers.

In some embodiments, for example, targeting refers to a method used to selectively amplify or otherwise preferentially enrich those molecules of DNA or RNA that correspond to a set of loci in a mixture of DNA or RNA.

### WORKING EXAMPLES

### Example 1

This example is illustrative only, and a skilled artisan will appreciate that the invention disclosed herein can be practiced in a variety of other ways.

### Blood Samples

Pregnant female adult or young-adult patients receive a donor organ from related or unrelated living donors, or unrelated deceased donors. Time points of patient blood draw following transplantation surgery are either at the time of an transplant biopsy or at various pre-specified time intervals based on lab protocols. Optionally, samples are biopsy-matched and blood are drawn at the time of clinical dysfunction and biopsy or at the time of protocol biopsy (at which time most patients do not have clinical dysfunction). In addition, some patients have serial post transplantation blood drawn.

### Nucleic Acid Measurement in Blood Samples

The workflow and statistical analysis disclosed in Sigdel et al., J. Clin. Med. 8(1): 19(2019 ) is incorporated herein by reference in its entirety. Nucleic acids such as RNA or DNA, and in particular cfDNA is extracted from plasma samples using the QIAamp™ Circulating Nucleic Acid Kit (Qiagen) and the LabChip™ NGS 5k kit (Perkin Elmer, Waltham, MA, USA) is used for quantification. Library preparation is performed using the Natera Library Prep kit as described in Abbosh et al, Nature 545:446-451 (2017), which is incorporated herein by reference in its entirety, with a modification of 18 cycles of library amplification to plateau the libraries. Purified libraries are quantified using LabChip™ NGS 5k as described in Abbosh et al, Nature 545:446-451 (2017). Target enrichment is accomplished using massively multiplexed-PCR (mmPCR) using a modified version of Zimmermann et al., Prenat. Diagn. 32:1233-1241 (2012), which is incorporated herein by reference in its entirety, with 13,392 single nucleotide polymorphisms (SNPs) targeted. Amplicons are then sequenced on an Illumina HiSeq 2500 Rapid Run®, 50 cycles single end, with 10-11 million reads per sample.

### Example 2

### Determination of Transplant State in Maternal Transplant Recipients

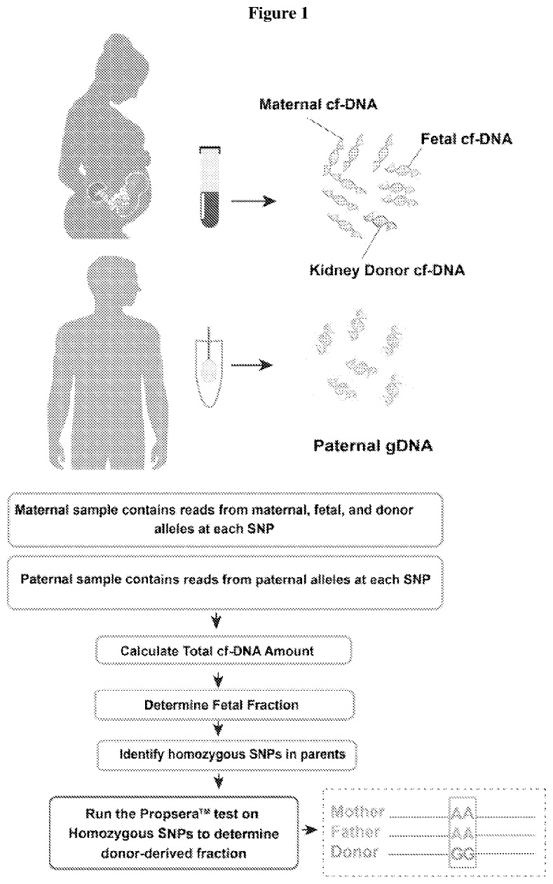

dd-cfDNA was measured in 6 pregnant kidney recipients as shown in FIG. 1. Identification of SNPs at which both the mother and father were homozygous ensured homozygosity of the fetus and that heterozygosity observed in the maternal cfDNA originated from the transplant. These SNPs were used to calculate donor fraction estimates (DFE). Clinical data collected included kidney function test, mode of delivery, preeclampsia, and preexisting hypertension.

DFE was calculated for 4/6 pregnant kidney transplant recipients. 5/6 of these patients had preexisting chronic hypertension and 4/6 had superimposed preeclampsia. All patients underwent medically indicated inductions of labor and subsequent cesarean births for varied indications. Chart review also revealed that all patients experienced a spike in serum creatinine and acute kidney injury during the study either before, after, or during delivery.

This study demonstrates that dd-cfDNA can be measured in pregnant kidney recipients that have chronic kidney dysfunction. These measurements can help differentiate between acute rejection and hypertensive diseases of pregnancy. A larger cohort study will confirm the utility of longitudinal monitoring during pregnancy to help inform the physician in clinical decision making that can affect both transplant outcomes and maternal fetal health.

### Example 3

### Donor-Derived Cell-Free DNA (dd-cfDNA) in Pregnant Kidney Transplant Recipients

Background. Symptoms of early preeclampsia and renal transplant rejection are indistinguishable in a non-invasive manner in pregnant kidney transplant recipients (KTRs). Donor-derived cell-free DNA (dd-cfDNA) is a biomarker that can be non-invasively measured to assess risk of allograft rejection. The Prospera™ test uses a single nucleotide polymorphism (SNP) based massively multiplexed PCR (mmPCR) methodology to measure dd-cfDNA in KTRs. Detection of dd-cfDNA ≥1% is associated with rejection in adult non-pregnant KTRs. Measuring dd-cfDNA in pregnant KTRs requires distinguishing donor cfDNA fragments from fetal and maternal cfDNA fragments. We have successfully demonstrated measurement of dd-cfDNA in a small cohort of pregnant KTRs with hypertensive disease and describe here two of the cases with medical details.

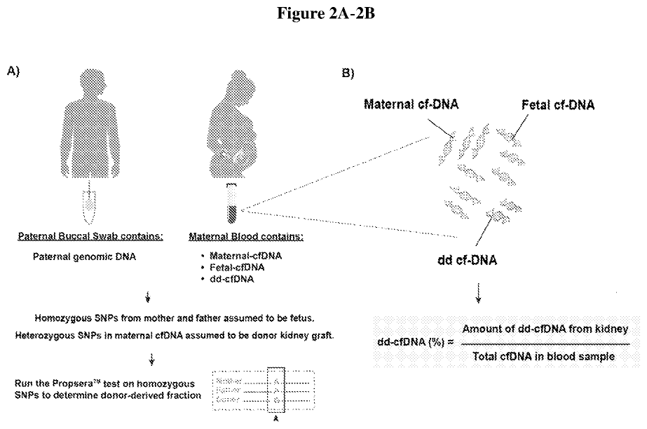

Method. Six pregnant KTRs were included in this study. Genomic DNA (gDNA) from the father and total cfDNA from the mother were extracted from buccal swab and blood draw, respectively (FIG. 2A). dd-cfDNA fraction was measured in the pregnant KTRs using an integrated (Panorama™/Prospera™) bioinformatics and lab workflow that incorporates sequencing of total maternal cfDNA and paternal gDNA (FIG. 2B). Clinical data was collected including kidney function test, mode of delivery, serum creatinine, preeclampsia, preexisting hypertension, and clinical history.

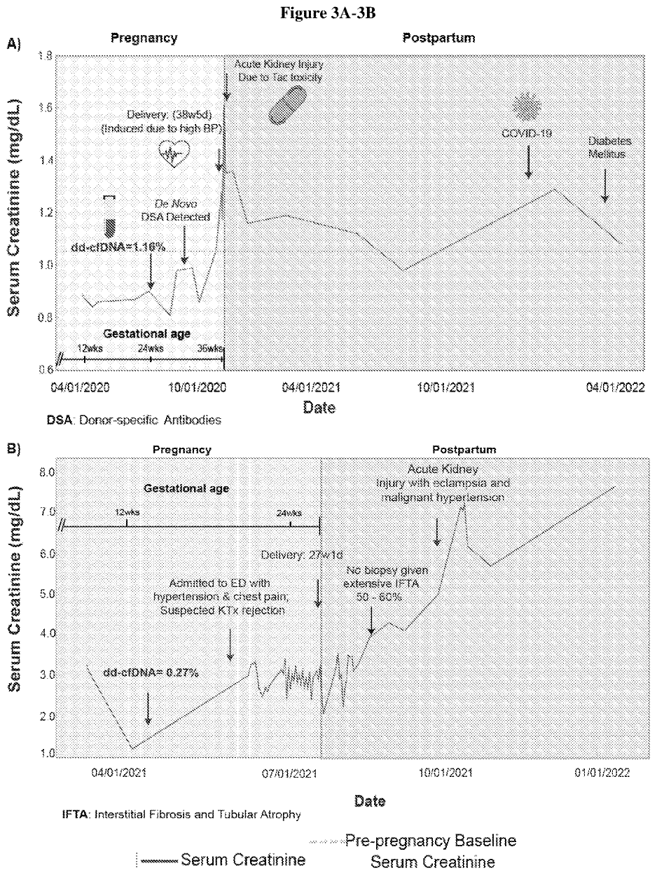

Results. dd-cfDNA was calculated for 4/6 pregnant KTRs; 5/6 patients had preexisting chronic hypertension and 4/6 had superimposed preeclampsia. All patients underwent medically indicated inductions of labor and subsequent cesarean births for varied indications. All patients experienced a spike in serum creatinine (SCr) and acute kidney injury (AKI) during the study either before, after, or during delivery. Patient 1 (FIG. 3A) was diagnosed with AKI due to tacrolimus toxicity on prior to conception and underwent labor induction at gestational age 38w5d due to high blood pressure and rising SCr levels. Three months prior to the AKI diagnosis, the patient had a dd-cfDNA of 1.16%. Patient 2 (FIG. 3B) SCr levels drastically increased throughout the course of pregnancy and postpartum. During pregnancy, the patient was suspected of having acute KT rejection with hypertension and had a dd-cfDNA of 0.27% around gestational age 15w. Complications with possible KT rejection prompted labor induction at gestational age 27w1d. Postpartum, the patient was diagnosed with AKI with eclampsia and malignant hypertension due to nonadherence.

Conclusion. This proof-of-concept study demonstrated that dd-cfDNA can be measured in pregnant KTRs that have chronic kidney dysfunction. These measurements help differentiate between acute rejection and hypertensive diseases of pregnancy. Further validation of dd-cfDNA testing in pregnant KTRs will help inform the physician of allograft-and maternal-fetal-health.

### Example 4

### Donor-Derived Cell-Free DNA (dd-cfDNA) in Pregnant Kidney Transplant Recipients

Successful kidney transplant restore fertility and enable childbearing aged kidney recipients to successful pregnancy. Pregnant people with a kidney transplantation are at higher risk for maternal fetal complications including miscarriage, preterm delivery, hypertensive disease of pregnancy as well as deterioration of graft function and rejection, and the rate of kidney transplantation in this population continues to grow. Graft function is imperative for both the pregnant person and the neonate, as well as the life course of the allograft. During the pregnancy, the ability to distinguishing between preeclampsia and kidney rejection transplantation is crucial, as the management, birth timing, and implications of the diagnosis differ dramatically.

The definitive diagnostic test for kidney rejection requires a renal biopsy, which is only recommended if a histological diagnosis will change management in pregnancy. Traditionally, the monitoring of kidney allografts involved assessing serum creatinine levels, as well as monitoring blood pressure and evaluating proteinuria. These parameters serve as indirect indicators of changes in allograft function and can suggest the possibility of acute rejection. However, those parameters are a relatively nonspecific marker and can be affected by factors other than rejection, such as dehydration, gestational age, and preeclampsia. Recently, donor-derived cell-free DNA (dd-cfDNA) has arisen as a non-invasive specific biomarker for allograft rejection.

Distinguishing between allograft rejection, kidney disease progression, and preeclampsia in pregnant kidney transplant recipients without resorting to kidney biopsy is not currently feasible in a non-invasive manner. Utilizing dd-cfDNA to monitor allograft status in pregnant kidney transplant recipients (KTRs) requires the ability to distinguishing between fetal/placental, donor, and recipient cfDNA fragments. This study aims to measure donor derived-cfDNA in a cohort of pregnant kidney transplant recipients using a combination of placentally derived cfDNA and transplant monitoring technology utilizing dd-cfDNA in conjunction with paternal DNA swabs. The study here demonstrate feasibility of utilizing dd-cfDNA in pregnancy from a cohort of pregnant people with hypertensive disease.

Method. The study included blood samples from pregnant people with a kidney transplant during pregnancy. Prospera™ (Natera, Inc.) was performed on pregnant patients with a kidney allograft (n=9). Prospera™ is a transplant rejection detection and surveillance test that detects allograft donor-derived, cell-free DNA (dd-cfDNA) in the blood. dd-cfDNA is expressed as a fraction of the total cell-free DNA (cfDNA). This clinical test is a massively-multiplexed PCR (mmPCR) single nucleotide polymorphism (SNP)-based genetic test that detects more than 13,000 SNPs to accurately measure dd-cfDNA. Prospera for kidney rejection uses a ≥1% cutoff of dd-cfDNA fraction to indicate active rejection. However, the donor fraction estimate calculation is more complex in a pregnant person because the cfDNA in the mother's peripheral blood is a mixture of mother's cfDNA, donor derived cfDNA from the transplanted organ and fetal cfDNA from maternal and paternal origin. To overcome this issue, the paternal genotype was first identified by sequencing a buccal swab from the father with a modified Prospera™ workflow with genomic DNA (gDNA) as input. Genomic DNA (n=6) was isolated using the Qiagen DNeasy Blood and Tissue Kit and quantified by Qubit dsDNA HS (Thermo). gDNA was sheared to approximately ~160 bp with the Covaris LE220 ultrasonicator. Sheared samples were quantified, normalized, and library prepared. After mmPCR, barcoded samples were pooled, quantified, and sized on the 2100 Bioanalyzer (Agilent) and sequenced on the NextSeq 500. The sequencing data was processed through a customized Prospera™ bioinformatic pipeline.

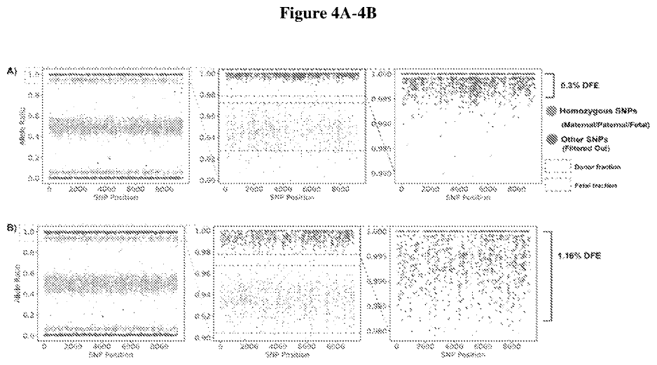

Determination of the donor derived cfDNA from the transplanted kidney. Donor-derived cell-free DNA (dd-cfDNA) was measured in 6 pregnant kidney recipients by sequencing the patient's cfDNA using the Prospera™ laboratory workflow starting from a peripheral blood sample (FIG. 2). A custom bioinformatics analysis was performed to quantify the donor derived cfDNA (FIG. 4). To determine the maternal genotype, any homozygous SNPs with greater than 75% of reads belonging to one allele are considered homozygous for the mother. To determine the paternal genotype, any SNPs from the paternal buccal swab with more than 99% of the reads belonging to one allele are considered homozygous. If both the maternal and paternal genotypes are homozygous for the same allele, then the fetal genotype must also be. After filtering for SNPs where the mother and the fetus have all four copies with the same allele, the Prospera™ algorithm is run on the filtered set of SNPs to determine the dd-cfDNA percentage (FIG. 4). Clinical data collected included kidney function test, mode of delivery, preeclampsia, and preexisting hypertension.

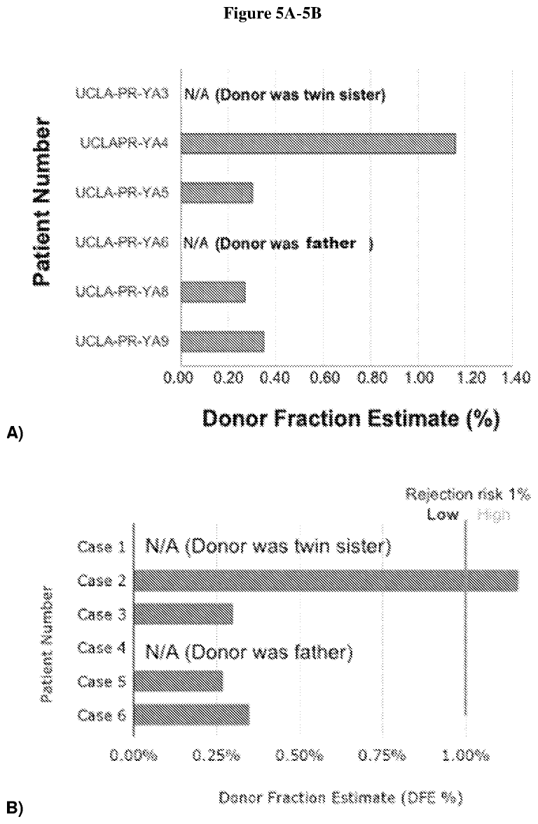

Results. Nine pregnant people were recruited. Paternal buccal swabs were received and available from six of the nine participants with known and confirmed paternity. These six participants were included in the study. Granular details are presented in Table 1. Two adjudicated representative cases are presented in FIG. 4. Donor-derived-cfDNA analysis using Prospera with a customized data analysis approach resulted in a Rejection Risk call for 4 patients (FIG. 5). In two cases, the donor cfDNA could not be distinguished because the donor was either the father of the fetus or the twin-sister of the recipient. From the 4 remaining cases, DFE was significantly below 1% in 3 cases, and therefore reported as low risk, but above the 1% threshold in case 2. Case 2 had also an unusually high total amount of cfDNA.

Discussion. Pregnancy in women undergoing dialysis is rare, with a low incidence of conception ranging from 0.9 to 7% and very high rates of preterm birth. Fertility may be compromised in these women due to worsening renal function, and kidney transplantation offers a restorative effect on ovulation and fertility. However, while most pregnancies following kidney transplantation result in live births, there is a significant risk of fetal complications, including preterm birth, low birth weight, and fetal growth restriction (FGR). The evidence on live birth rates among kidney transplant recipients who conceive is conflicting, with some studies showing comparable rates to the general population, while others report lower rates. During pregnancy, it is crucial to maintain adequate immunosuppression and adjust according to pharmacokinetic changes and physiological changes related to pregnancy.

For those reasons, close monitoring by both the transplant nephrologist and high-risk obstetrician is recommended for kidney transplant recipients during pregnancy. The monitoring should encompass surveillance for hypertension, preeclampsia, gestational diabetes, kidney allograft dysfunction, and infection, aiming for the primary goal of achieving a near or full-term pregnancy without hypertensive complications, graft dysfunction, or rejection.

The use of cell-free DNA (cfDNA) in prenatal care has gained significant interest in recent years. In the context of pregnancy, cfDNA, also known as non-invasive prenatal testing, enables the detection of fetal aneuploidy (specifically trisomy 21, 18 and 13 and sex chromosomes). Additionally, monitoring donor-derived cfDNA (dd-cfDNA) has shown promise as a non-invasive method to detect early signs of graft rejection in transplant recipients. Studies have indicated that the probability of acute kidney rejection increases when the dd-cfDNA level exceeds one percent.

Given these observations, it is reasonable to hypothesize that there is a correlation between the fraction of dd-cfDNA and the occurrence of rejection in pregnant kidney recipients, and by serial monitoring of dd-cfDNA levels, it might be possible to identify individuals at higher risk for these complications, enabling closer surveillance and timely interventions, and potentially serving as a helpful tool for differentiation of acute rejection from preeclampsia without the need for renal biopsy. This study successfully measured dd-cfDNA in a cohort of pregnant kidney transplant recipients using transplant monitoring technology utilizing dd-cfDNA and paternal buccal DNA swabs. In 2 cases the donor was the 2-haplotype sister of the mother or father of the child. In the 4 remaining cases 3 were determined as low risk for rejection and 1 was reported and possible high-risk (DFE >1%).

In conclusion, this study demonstrates that dd-cfDNA can be measured in pregnant kidney recipients and differentiate kidney rejection from hypertensive disease of pregnancy in a specific high-risk population of kidney transplant recipients. This non-invasive surrogate blood marker can help to differentiate acute rejection and hypertensive diseases of pregnancy. The presence of cfDNA in pregnant people has shown potential as a diagnostic and monitoring tool for transplant rejection. Exploring the correlation between the fraction of cfDNA and the occurrence of rejection in pregnant women with kidney transplants holds promise for improving risk assessment and patient management. This study may have a significant impact on the nearly ~3,500 women of reproductive age with solid organ transplants in the United States.

What is claimed is:

1. A method of preparing a composition of amplified DNA derived from a biological sample of a maternal transplant recipient useful for determination of transplant status, comprising:
(a) extracting cell-free DNA (cfDNA) from the biological sample of the maternal transplant recipient, wherein the extracted cfDNA comprises donor-derived cell-free DNA (dd-cfDNA) from the transplant, recipient-derived cell-free DNA (rd-cfDNA) from the maternal transplant recipient, and fetal-derived cell-free DNA (fd-cfDNA) from a fetus;
(b) preparing a composition from the cfDNA wherein a plurality of target loci are enriched, wherein the target loci comprise one or more SNP loci at which the maternal transplant recipient and biological father of the fetus are homozygous to ensure homozygosity of the fetus at the SNP loci, such that heterozygosity observed in the extracted cfDNA at the SNP loci originates from the transplant; and
(c) quantifying the amount of dd-cfDNA based on heterozygosity at the SNP loci to determine whether the amount of dd-cfDNA or a function thereof exceeds a cutoff threshold indicating transplant rejection.

2. The method of claim 1, wherein quantifying the cfDNA and dd-cfDNA comprises preparing a sequencing library from the extracted cfDNA and sequencing the sequencing library by high-throughput sequencing to obtain sequencing reads.

3. The method of claim 1 or claim 2, wherein the target loci comprises 10-50,000 target loci, and the method further comprises performing multiplex targeted amplification of the DNA at the 10-50,000 target loci in a single reaction volume.

4. The method of claims 1-3, wherein step (c) further comprises determining an amount of a transplant-derived allele at the one or more SNP loci, and determining whether the amount of the transplant-derived allele at the one or more SNP loci or a function thereof exceeds a cutoff threshold indicating transplant rejection; wherein transplant rejection is determined by a combination of (i) the amount of the transplant-derived allele at the one or more SNP loci or a function thereof, and (ii) the total amount of dd-cfDNA or the fraction of dd-cfDNA.

5. A method of administrating immunosuppressive therapy in a maternal transplant recipient, comprising:
(a) quantifying the total amount of cfDNA and the amount of dd-cfDNA in a biological sample of the transplant recipient according to the method of any of claims 1-5; and
(b) titrating the dosage of an immunosuppressive therapy according to the amount of cfDNA or a function thereof and the amount of dd-cfDNA or a function thereof.

6. The method of claim 5, further comprising repeating step (a) longitudinally for the same transplant recipient, and determining a longitudinal change in the amount of cfDNA or a function thereof and a longitudinal change in the amount of dd-cfDNA or a function thereof.

7. The method of claim 6, further comprising titrating the dosage of the immunosuppressive therapy according to the longitudinal change in the total amount of cfDNA or a function thereof and the longitudinal change in the amount of dd-cfDNA or a function thereof.

8. The method of any of claims 5-7, wherein:
an increase in the levels of dd-cfDNA are indicative of transplant rejection and a need for adjusting immunosuppressive therapy; and
change or a decrease in the levels of dd-cfDNA indicates transplant tolerance or stability, and a need for adjusting immunosuppressive therapy.

9. The method of any one of claims 1-8, further comprising sequencing paternal DNA of the biological father of the fetus and identifying the one or more SNP loci at which the maternal transplant recipient and biological father of the fetus are homozygous while the dd-cfDNA comprises a heterozygous allele.

10. The method of any one of claims 1-9, wherein the method is performed without prior knowledge of donor and/or recipient genotypes.

11. £ The method of any of claims 1-10, further comprising performing universal amplification of the extracted DNA.

12. The method of claim 11, wherein the universal amplification step preferentially amplifies dd-cfDNA over rd-cfDNA and fd-cfDNA.

13. The method of any of claims 1-12, wherein the extracting step comprises size selection to enrich for dd-cfDNA and reduce the amount of rd-cfDNA and fd-cfDNA.

14. The method of any of claims 1-13, wherein the amount of cfDNA is measured by quantitative PCR, real-time PCR, digital PCR, sequencing, microarray, or molecular barcodes and microscopic imaging.

15. The method of any one of claims 1-14, wherein the amount of dd-cfDNA is determined by using ratiometric and/or machine learning-artificial intelligence comparisons at a single or a plurality of time points.

16. The method of any one of claims 1-15, wherein the cutoff threshold is an estimated percentage of dd-cfDNA out of total cfDNA or a function thereof.

17. The method of any of claims 1-16, wherein the amount of dd-cfDNA of greater than 1% of total cfDNA indicates that the transplant is undergoing acute rejection, and wherein an amount of dd-cfDNA of less than 1% of total cfDNA indicates that the transplant is either undergoing borderline rejection, undergoing other injury, or is stable.

18. The method of any one of claims 1-17, wherein the transplant recipient has received one or more transplants selected from kidney, liver, pancreas, intestinal, heart, lung, heart/lung, stomach, testis, penis, ovary, uterus, thymus, face, hand, leg, bone, bone marrow, cornea, skin, pancreas islet cell, heart valve, blood vessel, and blood transfusion.

19. The method of any one of claims 1-18, wherein the sample is obtained from the transplant recipient less than 18 months post-transplantation.

20. The method of any one of claims 1-19, wherein the rejection risk for the transplant recipient is determined using logistic regression, random forest, or decision tree machine learning analysis.

21. The method of claim 20, wherein the logistic regression, random forest, or decision tree machine learning analysis further incorporates one or more parameters selected from time post-transplantation, age of transplant recipient and/or transplant donor, gender of transplant recipient and/or transplant donor.

22. The method of any of claims 1-21, wherein the biological sample is blood, serum, plasma, or urine.
